(19) United States
(12) Patent Application Publication
(10) Pub. No.: US 2019/0344084 A1
(43) Pub. Date: Nov. 14, 2019

(54) MEDICAL DEVICE INCLUDING TOOL-GRIPPING PORTION

(71) Applicant: INSPIRE MEDICAL SYSTEMS, INC., Golden Valley, MN (US)

(72) Inventors: KEVIN VERZAL, Golden Valley, MN (US); John Rondoni, Golden Valley, MN (US); Jordan McIver, Golden Valley, MN (US); Luke Lozier, Golden Valley, MN (US); Sean Buckner, Golden Valley, MN (US)

(73) Assignee: INSPIRE MEDICAL SYSTEMS, INC., Golden Valley, MN (US)

(21) Appl. No.: 16/408,670

(22) Filed: May 10, 2019

Related U.S. Application Data
(60) Provisional application No. 62/669,658, filed on May 12, 2018.

(51) Int. Cl. A61N 1/375; A61N 1/05; A61N 1/372

(52) U.S. Cl. CPC A61N 1/375; A61N 1/37205; A61N 1/0556; A61N 1/0504

(57) ABSTRACT

A medical device comprises a non-gripping portion and a tool-gripping portion.

### CROSS-REFERENCE TO RELATED APPLICATION

This application is a utility application which claims the benefit of U.S. Patent Application Ser. No. 62/669,658 filed May 12, 2018, and incorporated herein by reference.

### BACKGROUND

Implanting or delivering medical devices within a patient's body often involves the use of tools to gain access to the body, create tunnels, and the like.

### BRIEF DESCRIPTION OF THE DRAWINGS

FIG. 1 is a top plan view schematically representing an example medical device.

FIG. 2A is a top plan view schematically representing an example implantable lead.

FIG. 2B is an enlarged partial top plan view schematically representing a first portion and anchor of an example implantable lead.

FIG. 2C is an enlarged partial side plan view schematically representing a first portion and anchor of an example implantable lead.

FIG. 3 is an enlarged partial Feature Setal view schematically representing an example tool-gripping portion and first portion of an example implantable lead.

FIG. 4 is an enlarged partial Feature Setal view schematically representing a coating layer on a first portion of an example implantable lead.

FIG. 5A is a diagram schematically representing a patient's ribcage and an example implantable lead.

FIG. 5B is a diagram schematically representing an example implantation of an example implantable lead.

FIGS. 6A-6C are a series of diagrams schematically representing an example implantation of an example implantable lead via a tool in association with a tool-gripping portion.

FIGS. 7-8 are each a diagram including an enlarged side view schematically representing an example implantation of an example implantable lead via a tool in association with a tool-gripping portion.

FIGS. 9A-9D are a series of diagrams schematically representing an example implantation of an example implantable lead via a tool in association with a tool-gripping portion of the lead.

FIG. 10 is a diagram, including a side Feature Setal view, schematically representing an example implantation within tissue of a first portion of an example implantable lead.

FIG. 11 is an enlarged partial Feature Setal view schematically representing an example tool-gripping portion of an example implantable lead.

FIG. 12 is an enlarged partial Feature Setal view schematically representing at least some components of an example tool-gripping portion and sensor portion of an example implantable lead.

FIG. 13A is an enlarged Feature Setal view schematically representing a coil structure of an example tool-gripping portion of an implantable lead.

FIG. 13B is an enlarged Feature Setal view schematically representing a coating layer on the example coil structure of FIG. 13A.

FIG. 14A is an isometric view schematically representing an example tool.

FIG. 14B is an enlarged partial isometric view schematically representing an example tool engaging a medical device.

FIG. 14C is an end view schematically representing an example tool.

FIG. 15A is an isometric view schematically representing an example tool.

FIG. 15B is an enlarged partial isometric view schematically representing an example tool engaging a medical device.

FIG. 15C is an end view schematically representing an example tool.

FIGS. 16A-16F are a series of diagrams schematically representing an example method of implanting a lead.

FIGS. 17A-17F are a series of diagrams schematically representing an example method of manufacturing of an implantable lead.

FIGS. 18A-18E are a series of top plan view diagrams schematically representing example implantable leads.

FIG. 19A is a flow diagram schematically representing an example method.

FIGS. 19B-19E are each a diagram schematically representing an example implantable medical device.

### DETAILED DESCRIPTION

In the following detailed description, reference is made to the accompanying drawings which form a part hereof, and in which is shown by way of illustration specific examples in which the disclosure may be practiced. It is to be understood that other examples may be utilized and structural or logical changes may be made without departing from the scope of the present disclosure. The following detailed description, therefore, is not to be taken in a limiting sense. It is to be understood that features of the various examples described herein may be combined, in part or whole, with each other, unless specifically noted otherwise.

At least some example medical devices are directed to providing a tool-gripping portion to facilitate delivery of the medical device relative to a patient's body. In some examples, the medical device may be internally delivered within the patient's body for some temporary purpose and then later withdrawn. In some examples, the medical device is implantable and is intended to remain implanted in the patient's body for a prolonged period of time.

In some examples, the tool-gripping portion may comprise a visual designation to enhance visual recognition of the tool-gripping portion and visually distinguishing the tool-gripping portion from other non-gripping portions of the medical device and/or from the environment (e.g. blood, tissue, etc.) within the patient's body. In some such examples, via this arrangement more repeatable outcomes may be achieved when internally delivering a medical device within a patient's body. Moreover, this arrangement may enhance surgical effectiveness and efficiency. In some such examples, at least some of the non-gripping portions of the medical device may comprise more sensitive structures, components, etc. such that use of the tool-gripping portion for handling the medical device helps to protect the structural integrity of the non-gripping portions of the medical device. This protection occurs at least because any tool-gripping pressure or handling of the lead will be located away from the non-gripping portions. In some instances, the tool-gripping portion may sometimes be referred to as a tool-gripping zone.

In some examples, the medical device may comprise a flexible, resilient elongate body including a tool-gripping portion and a non-gripping portion. In some such examples, the tool-gripping portion may be adjacent the non-gripping portion. In some examples, the non-gripping portion may comprise an operative element to interact in some manner within a patient's body. In some examples, the operative element may implement an interaction such as sensing, stimulation, monitoring, delivery of fluids, etc. In some such examples, the medical device may comprise an implantable lead, such as but not limited to, sensor leads, stimulation leads, and the like. In some such examples, the sensor lead may comprise a lead for sensing respiratory information and/or other physiologic information.

In some examples, the operative element for the sensor lead may comprise a pressure-indicative sensor for sensing at least respiratory information. In some examples, the implantable medical device may comprise a catheter in which the operative element in the distal portion may be used for drug delivery or other purposes.

In some examples, the medical device may comprise at least two tool-gripping portions. In some examples, the non-gripping segment may be interposed between two spaced apart tool-gripping portions.

In some examples, the non-gripping portion (which may comprise an operative element) comprises a distal component from which the tool-gripping portion extends proximally adjacent the non-gripping portion.

In some examples, the tool-gripping portion is immediately adjacent (e.g. borders) the non-gripping portion. In some examples, the tool-gripping portion is spaced apart, along at least a portion of the length of the medical device, from the non-gripping portion.

In some examples, the tool-gripping portion of the medical device may comprise an increased thickness of electrically non-conductive material, which enhances durability of at least some portions of the medical device when subject to handling by tools.

In some examples, the increased thickness of the electrically non-conductive material in the tool-gripping portion provides a larger surface area about which a gripping force exerted via a tool may be distributed, which thereby may decrease the amount of localized pressure transmitted to conductive structures underlying the electrically non-conductive material, thereby protecting their integrity. In addition, in some examples, the relatively larger surface area of the tool-gripping portion also provides a larger target on which gripping tool may be releasably engaged, to increase the sureness of gripping and/or the ease in establishing a gripping position. In addition, the use of a tool-gripping portion and/or the electrically non-conductive material forming at least part of the tool-gripping portion (as described in at least some of the examples of the present disclosure) also may enhance protection of any internal lumens (extending within a medical device on which the tool-gripping portion is mounted), may enhance kink-resistance, may further ensure maintaining connectedness of distal tip portions, and/or provide additional electrical insulation.

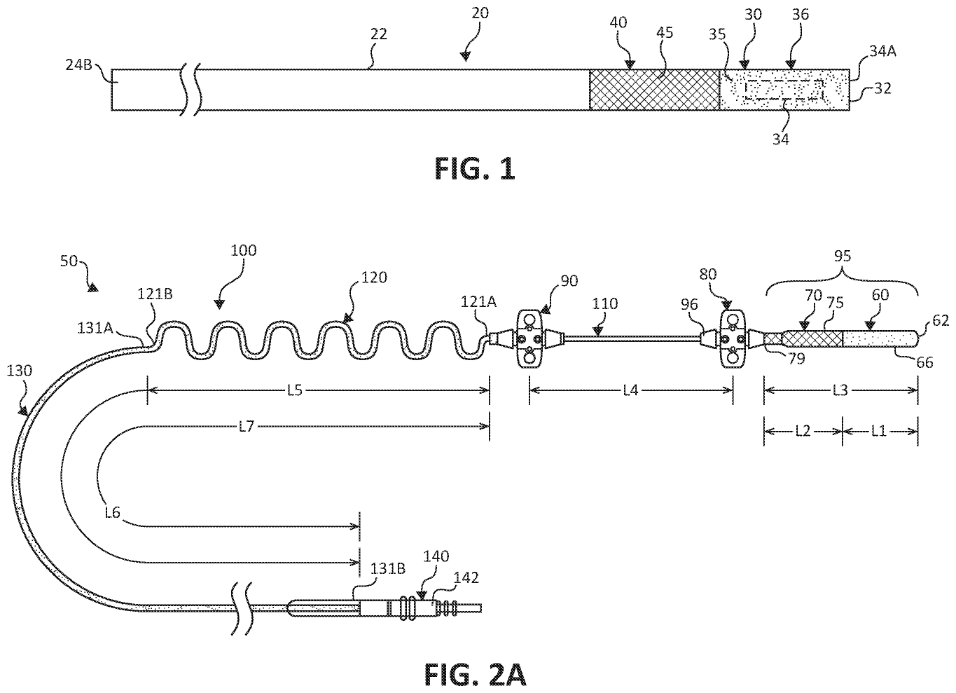

These examples, and additional examples, are described in greater detail in association with at least FIGS. 1-19E.

FIG. 1 is a side plan view schematically representing an example medical device 20. In some examples, the medical device 20 is delivered relative to a patient's body. As shown in FIG. 1, the medical device 20 comprises a non-gripping portion 36 and a distal tip 32. The medical device 20 comprises an elongate, flexible resilient body 22. In some examples, the body 22 extends proximally from the non-gripping portion 36 and in some examples, the body 22 at least partially defines the non-gripping portion 36. In some examples, the medical device 20 comprises a tool-gripping portion 40 adjacent the non-gripping portion 36 and which may be at least partially formed via the body 22. In some examples, such as shown in FIG. 1, the tool-gripping portion 40 is proximal to, and immediately adjacent the non-gripping portion 36. In some such examples, the non-gripping portion 36 comprises a distal portion of the medical device 20. The medical device 20 comprises a length extending between a distal end 24A and an opposite proximal end 24B.

The tool-gripping portion 40 facilitates gripping a portion of the medical device 20 without placing gripping pressure or direct handling of the non-gripping portion 36, thereby protecting any sensitive components, arrangements within or on the non-gripping portion 36.

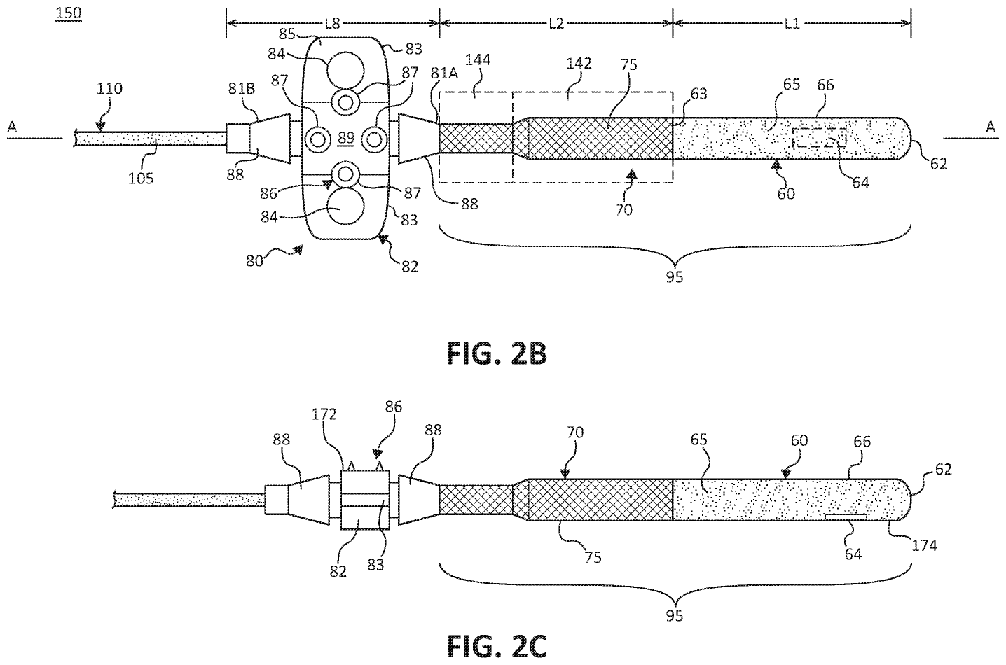

In some examples, the tool-gripping portion 40 comprises a first visual designator 45 while the non-gripping portion 36 may comprise a second visual designator 35 in some examples. Further details regarding at least some examples of the first visual designator 45 and second visual designator 35 are provided below in association with FIGS. 2-19E.

In some such examples, the non-gripping portion 36 comprises an operative element 34 to perform or facilitate some interaction between an environment of a patient's body and the operative element 34. It will be understood that the operative element 34 may comprise a single component or multiple components. In some examples, the operative element 34 may comprise at least one of a mechanical function element, an electrical function element, a chemical function element, and a thermal function element. In some such examples, the chemical function element may comprise a drug delivery mechanism as described in further detail herein.

In some examples, the medical device 20 may be internally delivered within a patient's body. In some such examples, the medical device may remain in the body temporarily, while in some such examples, the medical device may remain in the body for extended period of time.

In some examples, the medical device 20 comprises an implantable lead. In some examples of the implantable lead, the operative element 34 comprises at least one of a sensing element and a stimulation element. At least some specific examples of an implantable lead are described further below in association with at least FIGS. 2-19E.

In some examples, the medical device 20 comprises a catheter. In some such examples, the operative element 34 of the catheter may comprise a drug delivery mechanism or other fluid delivery mechanism. In some examples, the catheter is internally deliverable within a patient's body.

FIGS. 2A-13 schematically represent one example of medical device 20 in FIG. 1 when implemented as an implantable lead 50 including a distal portion 60 (e.g. a sensor portion) comprising an operative element 64 implemented as a sensing element, and with lead 50 including a tool-gripping portion 70 proximally adjacent the first portion 60. However, it will be understood that the at least some of substantially the same features and attributes of the implantable lead 50, with respect to at least the tool-gripping portion 70 and/or first portion 60, may be implemented as part of a medical device other than an implantable lead, such as a catheter or other device as previously mentioned. In such examples, the operative element 64 may comprise a drug delivery mechanism, a thermal element, stimulation element, etc.

FIG. 2A schematically represents an example implantable lead 50. In some examples, the implantable lead 50 may comprise a sensor lead. However, in some examples the lead 50 may comprise solely a stimulation lead in which operative element 64 comprises a stimulation element or may comprise a lead in which operative element(s) 64 comprises both sensing and stimulation elements. In some examples, operative element 64 may comprise elements other than a sensing element or a stimulation element.

As shown in FIG. 2A, the lead 50 may comprise a first portion 60 extending distally from a lead body 100. In some examples, the first portion 60 may sometimes be referred to as a distal portion. In some examples, the first portion 60 may sometimes be referred to as a sensor portion when the operative element 64 comprises at least a sensing element. In some examples, the first portion 60 may sometimes be referred to as a stimulation portion when the operative element 64 comprises at least a stimulation element.

The lead body 100 comprises a flexible, resilient elongate member having sufficient rigidity to be pushable, steerable, etc. such as during subcutaneous tunneling, implantation, etc. Meanwhile, in some examples the first portion 60 may comprise a generally rigid member, which may facilitate positioning and secure implantation within target tissue. In some examples, first portion 60 comprises a length L1, which may comprise about 1 to about 10 centimeters in some examples.

As further shown in FIG. 2A, in some examples the lead 50 may comprise a tool-gripping portion 70 proximally adjacent the first portion 60 and which has a length L2. In some examples, the tool-gripping portion 70 extends proximally directly from the first portion 60. In some examples, the first portion 60 and tool-gripping portion 70 together may comprise a length L3, which may be about 0.5 to about 5 centimeters in some examples.

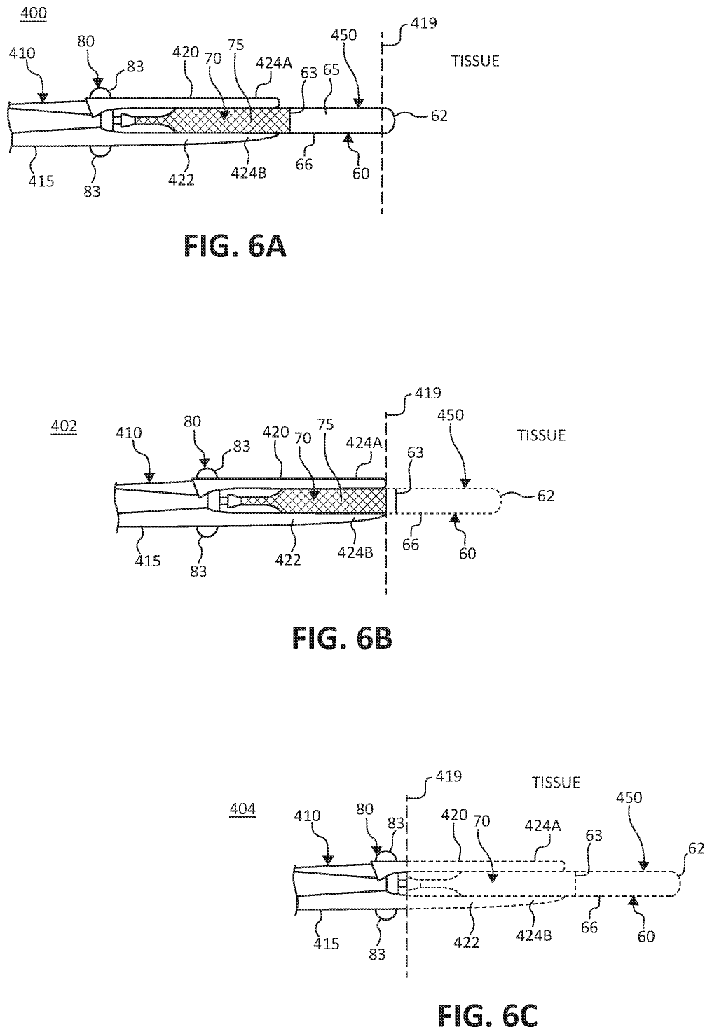

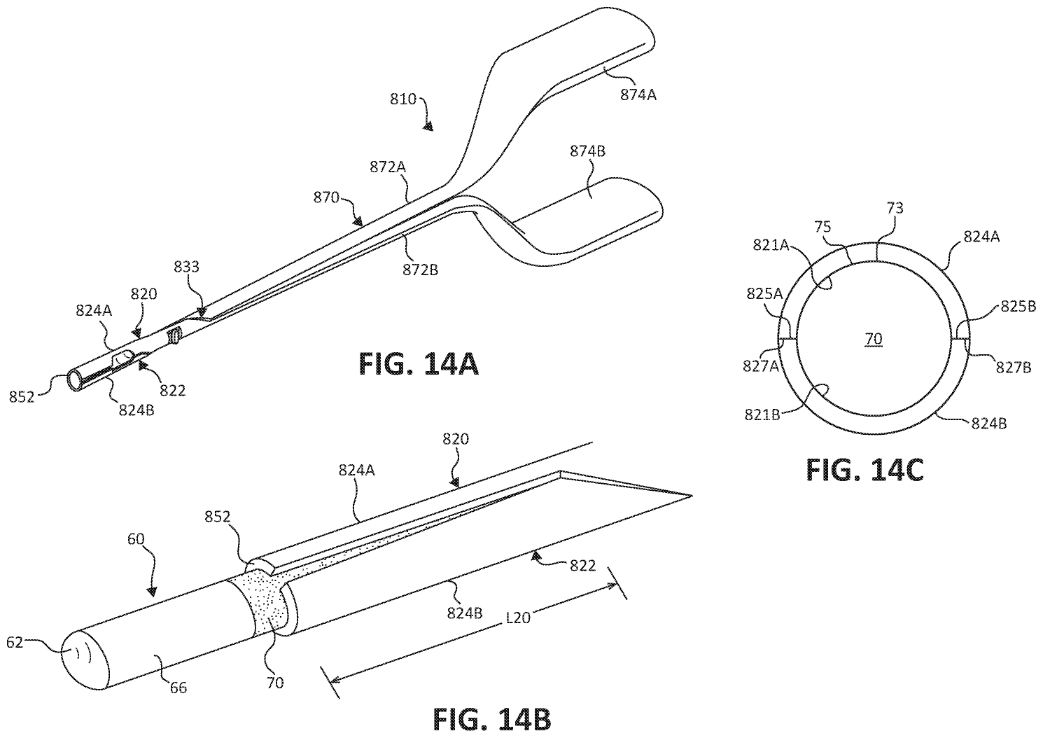

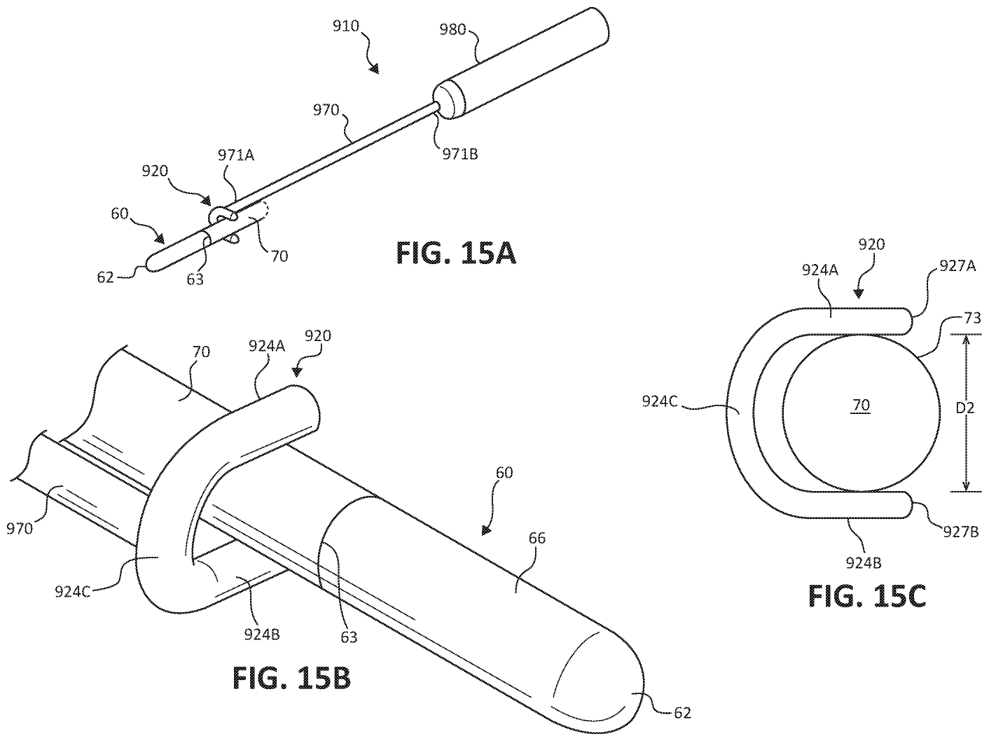

In some examples, the tool-gripping portion 70 may comprise a structure configured to be gripped by a tool, such as but not limited to, tool 410 as further illustrated later in association with at least FIGS. 6A-6C, 7, 8, and 9A-9D. In some examples, such a tool may take the form of one of the tools as described and illustrated later in association with at least FIGS. 14A-15C.

In some examples, the tool-gripping portion 70 may comprise a first visual designator 75 (as represented via a diamond pattern) to cause the tool-gripping portion 70 to be visually differentiated (e.g. identifiable) from the first portion 60 and/or from the first anchor 80. Stated differently, the first visual designator 75 enables the tool-gripping portion 70 to be perceptibly visually different from other portions of the lead 50 and/or from the environment within the patient's body. In some examples, the first visual designator 75 may be implemented as a color, a surface pattern, texture, a relative opaqueness (e.g. degree of transparency), reflectance, absorbance, relative radiopaqueness, topographic features, and/or profile. Such topographic features may comprise protrusions and/or recesses, which have a size, shape, and/or pattern suitable to visually distinguish the tool-gripping portion 70 from other portions (e.g. first portion 60, anchor portion 80) of the lead 50.

In some examples, the first visual designator 75 is distinguishable from the environment within the patient's body, such as blood, tissues, bone, fluids, etc.

In some examples, the first visual designator 75 may be implemented solely or primarily via a color. In some examples, the color is blue. In some examples, the color may be selected as being within a portion of the wavelength spectrum to be substantially visually perceptibly different from a portion of the wavelength spectrum of other portions of lead 50 and/or of the wavelength spectrum of elements within the environment of the patient's body. In some examples, the first visual designator 75 may comprise a composite color.

In some examples, the first visual designator 75 may comprise a combination of several different colors (which remain separate from each other) arranged in a pattern to be visually perceptibly different from a single color or combination of colors of other visual designators (e.g. 65, 85 in FIG. 2B) and/or visually perceptibly different from a single color or a combination of colors of other portions of a medical device or lead.

It will be understood that the diamond pattern shown in the FIGURES as first visual designator 75 does not (necessarily) bear any particular relationship to the actual structures, materials, etc. of the tool-gripping portion 70, and instead is provided for illustrative purposes to represent any one or more of the various types of above-described example implementations (e.g. color, texture, surface pattern, etc.) of a visual designator. At least some of these features associated with tool-gripping portion 70 are described in further detail in association with at least FIGS. 2A-2C.

In some examples, the first visual designator 75 extends a full length of the tool-gripping portion 70, while in some examples, the first visual designator 75 extends just a partial length of the tool-gripping portion 70.

In some such examples, the first visual designator 75 may additionally comprise, or alternatively comprise an additional thin sleeve which fits over the area (e.g. length) to function as the first visual designator 75. For instance, the sleeve may comprise a heat-shrink tube made of a polymer material (e.g. polytetrafluoroethylene PTFE) in some examples. Via this sleeve exhibiting a raised profile (e.g. increased thickness) relative to the non-gripping portion 66, the first visual designator 75 may be visually and/or tactilely perceptibly different from other portions of the lead 50, including the non-gripping portion 66.

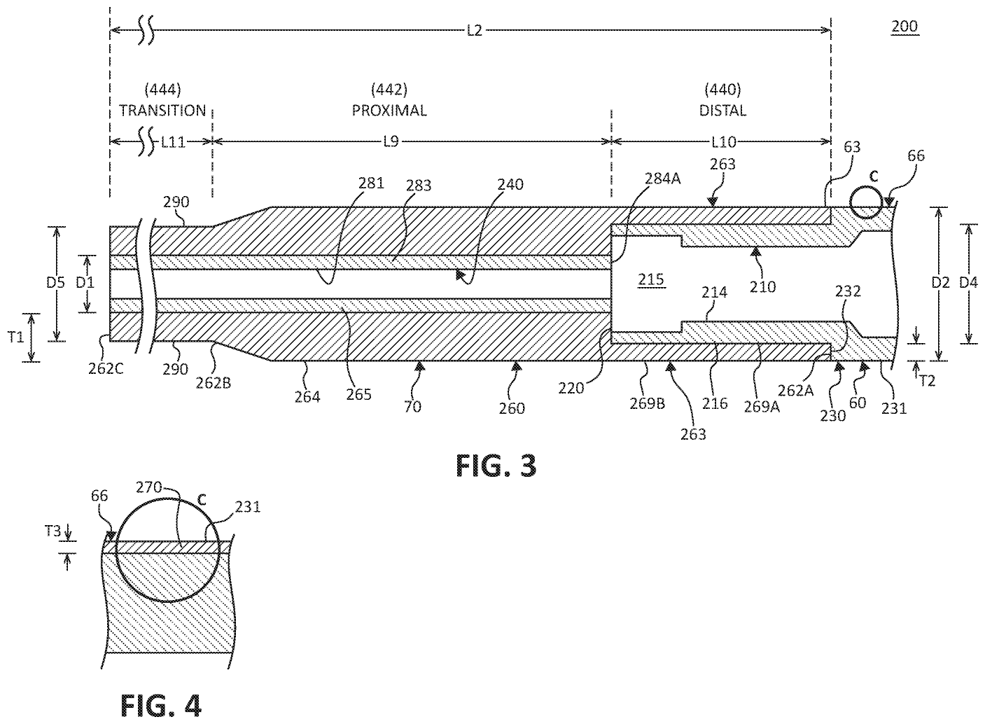

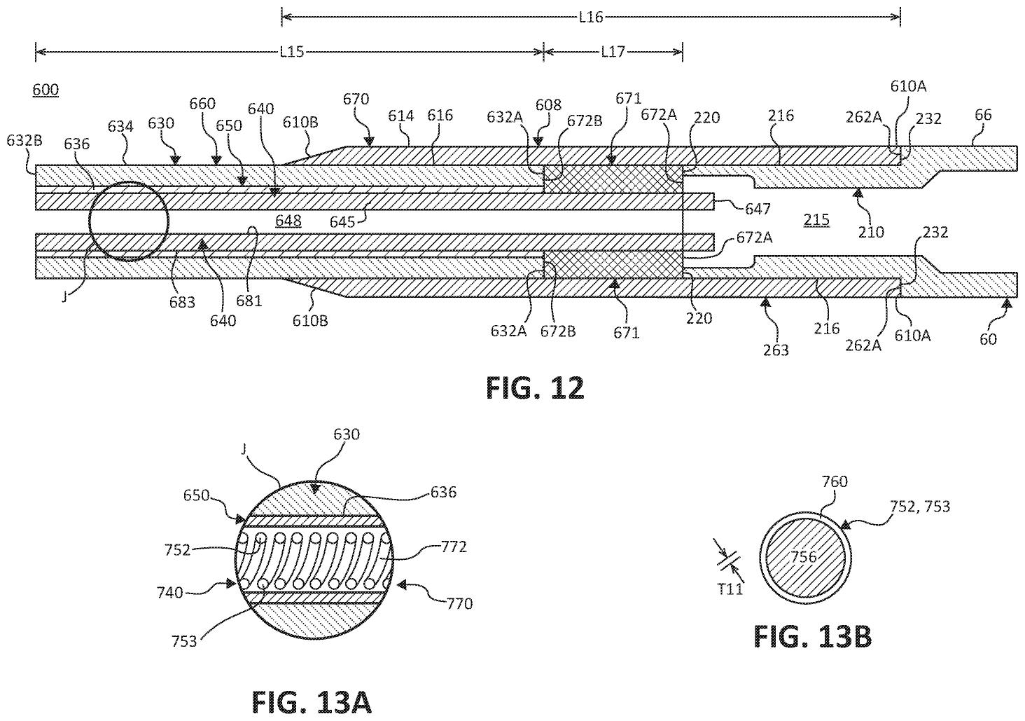

In some of the previously described examples of a first visual designator 75, the first visual designator 75 may additionally comprise, or alternatively comprise, an electrically conductive underlying structure (e.g. electrically conductive inner portion 240 (FIG. 3), 640 (FIGS. 12-13A) which is visible through a translucent or clear coating. In other words, the particular configuration and features of the underlying structure exhibits a visually perceptibly different appearance than other portions of the lead 50 (e.g. non-gripping portion 66) such that an operator can readily identify the tool-gripping portion 70, and/or differentiate the tool-gripping portion 70 from other portions of the lead, the environment (e.g. blood, tissue, bone, other body fluids, etc.) in the patient's body, etc.

In some examples, the first portion 60 may comprise a second visual designator 65 (as represented via a stippled pattern) to cause the first portion 60 to be visually differentiated from at least the first visual designator 75 of the tool-gripping portion 70. It will be understood that the stippled pattern shown in the FIGS as representing the second visual designator 65 does not necessarily bear any particular relationship to actual structures, materials, etc. of the first portion 60 (including non-gripping portion 66) but is provided for illustrative purposes to schematically represent the second visual designator 65 described herein.

In some examples, the second visual designator 65 may be implemented as a color, a surface pattern, texture, a relative opaqueness (e.g. degree of transparency), reflectance, absorbance, relative radiopaqueness, topographic features, and/or profile.

In some examples, the second visual designator 65 of first portion 60 may comprise a natural color and/or natural relative opacity of a non-conductive outer coating and natural color of an underlying conductive structure(s) of the first portion 60. For example, the natural color and relative opacity of the non-conductive outer coating may correspond to a clear, translucent material which reveals a stainless steel outer surface of the underlying conductive structures, which may have a grey or silver appearance. However, in some examples, the second visual designator 65 may comprise an additive material, added structure, and/or added treatment to make the structures and/or materials of the non-gripping portion 66 to be more visually distinctive alone and/or relative to the first visual designator 75 of the tool-gripping portion 70. In some such examples, in which both the second visual designator 65 of the non-gripping portion 66 and the first visual designator 75 of the tool-gripping portion 70 may comprise an electrically non-conductive outer portion (e.g. 260 in FIG. 3 or 270 in FIG. 4), the tool-gripping portion 70 may be visually perceptibly differentiated from the non-gripping portion 66 by, at least, a difference in the size, shape, configuration of the underlying electrically conductive structures of the respective tool-gripping portion 70 and non-gripping portion 66.

Accordingly, via such example arrangements, the first visual designator 75 of tool-gripping portion 70 may be readily identifiable by its contrast relative to at least the second visual designator 65 of first portion 60.

Moreover, in some instances, the first visual designator 75 associated with the tool-gripping portion 70 is selected to present a significant contrast to colors, textures, relative opacities, etc. of the various components (e.g. blood, tissue, body fluids, bone, etc.) within a patient's body which may be observed during implantation of lead 50 generally, and in particular, during implantation of the more distal portions of the lead, such as the first portion 60, tool-gripping portion 70, etc. In some such examples, the first visual designator 75 of tool-gripping portion 70 may comprise a color within the blue wavelength range, which may stand in sharp contrast to or be substantially different from a color of skin, underlying tissues, fat, muscle, blood, etc. within the patient's body. In some examples, in this context the term “substantially different” comprises a wavelength difference of 390-630 nanometers, spanning from orange thru violet in the visible light spectrum.

In some such examples, using a first visual designator 75 of a color within the blue wavelength range for tool-gripping portion 70 stands in vivid contrast to a first portion 60 (e.g. a sensor portion in one example) having a metal underlying structure, which is visible through a translucent, electrically non-conductive outer coating. In some such examples, the metal may comprise a stainless steel material, which may have a silver or grey appearance. In some such examples, the blue color wavelength range is substantially different from a 380-750 nanometer color wavelength range associated with the first portion 60.

In some examples, the lead 50 comprises a first anchor 80. In some such examples, the first visual designator 75 of a color within a wavelength range (for tool-gripping portion 70) stands in vivid contrast to a first anchor 80 comprising a third visual designation 85. In some examples, the first visual designator 75 comprises a color (e.g. blue) within a color wavelength range which is substantially different from a color (e.g. white) within a color wavelength range of the third visual designator 85.

In some examples, the lead 50 may comprise a second anchor 90. In some examples, both the first anchor 80 and second anchor 90 are fixed relative to, and along the length of, the lead body 100. The lead body 100 comprises a segment 110 extending between the two spaced apart anchors 80, 90. In some examples, anchors 80, 90 are spaced apart by a distance L4. In some examples, the distance L4 may comprise about 5 to 10 centimeters, and may be 7 centimeters, in some examples.

In some examples, second anchor 90 comprises at least some of substantially the same features and attributes as first anchor 80 in FIGS. 2B-2C.

In some examples, one or both of the respective first and second anchors 80, 90 may comprise a radiopaque material, which is readily identifiable when viewed via various imaging techniques. In some examples, the radiopaque material may comprise a white color, when viewed naturally (unaided via imaging tools). In some such examples, this white color comprises a vivid contrast to the visual designator 75 of tool-gripping portion 70. In some examples, a silicone tube may be fitted under each anchor 80, 90 to facilitate their fixation (to prevent both translation and rotation) relative to the lead body 100 during manufacturing and thereby avoid fixation (e.g. via suturing) during implantation.

In some examples, as later described in association with at least FIG. 12, a jacket may extend proximally from tool-gripping portion 70 and underneath first anchor 80, and slightly proximally beyond a proximal end of first anchor 80. In some examples, this jacket may provide abrasion-resistance at a location just proximal to the first anchor 80 in view of significant bending which may occur at this portion of the lead 50. In some examples, the jacket may comprise a flexible, resilient material, such as a polymer (e.g. polyurethane, silicone, etc.) in some examples.

In some examples, the lead body 100 comprises a resilient, flexible serpentine-shaped segment 120 extending a length L5, which comprises at least a portion of a full length L7 of the lead body 100 extending proximally from the second anchor 90. In one aspect, the serpentine-shape segment 120 may offer strain relief by allowing the lead body 100 to flex with various bodily movements of the patient, such as bending, twisting, leaning, stretching, etc. In some examples, the lead body 100 comprises a resilient, flexible linear (e.g. non-serpentine) segment 130 extending proximally from the serpentine-shaped segment 120 with segment 130 having a length L6. In some examples, the serpentine segment 120 extends a majority of the length L7 of the lead body 100 proximal to the second anchor 90. In some examples, the serpentine segment 120 comprises less than a majority of the length L7 of the lead body 100 proximal to the second anchor 90. In one aspect, the location and length of the serpentine segment 120 is selected so that upon implantation the serpentine segment 120 in areas of the patient's body (e.g. torso) which experience significant motion (e.g. twisting, bending, etc.), the serpentine shape may prevent tension on the lead body 100. In some such examples, the serpentine segment 120 may sometimes be referred to as a sigmoid segment. In some such examples, the serpentine segment 120 may be viewed as providing mechanical isolation between different portions of the lead 50. In some examples, the lead 50 may comprise at least two serpentine segments. In some examples, the lead 50 may comprise a serpentine segment (similar to segment 120) at other locations along the length of lead 50.

In some examples, any serpentine segments of the lead 50 may sometimes be referred to as a sigmoid-shaped segment, sinusoidal-shaped, segment, etc.

In some examples, portions of the lead body 100 proximal of the first anchor 80 are considered to be non-gripping portions of the lead 50, and as such are not recommended to be gripped by a tool, at least in the manner for which tool-gripping portion 70 is provided. In some such examples, these non-gripping portions proximal to first anchor do not have a visual designator (like designator 65) because these non-gripping portions do not include an operative element 64 and/or other sensitive structures.

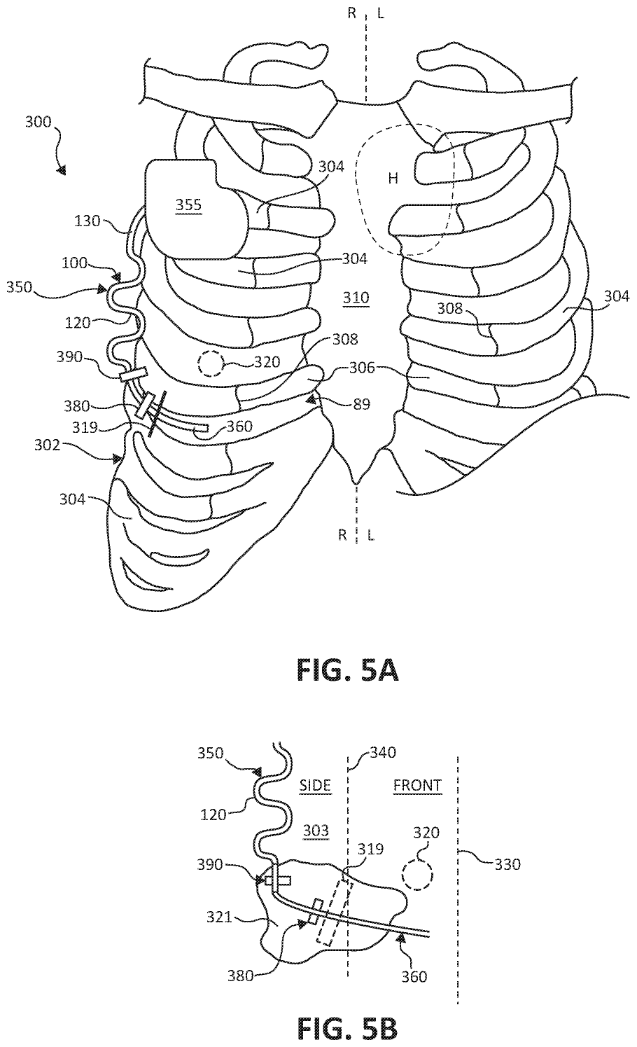

In some examples, the proximal portion 140 of the lead 50 comprises a connector portion 142 for removable connection to a port, such as a port of a sensor monitor, a generator, etc. One example of connection to a generator (e.g. 355) is shown later in at least FIG. 5A. In some examples, the generator 355 may comprise a pulse generator. In some examples, the generator 355 may be implantable within the patient's body.

In some examples, the first portion 60 may comprise a sensor(s) to sense respiratory-related information. In some such examples, the sensor(s) of the first portion 60 may comprise any one or more of a wide variety of modalities, such as but not limited to, electrical, chemical, mechanical, thermal, etc. and sense a wide variety of physiologic parameters, such as pressure, temperature, acoustics, posture, position, activity, respiration, cardiac information, etc. In some examples, at least some of these physiologic parameters may comprise information related to sleep disordered breathing, such as but not limited to, apneas, hypoponeas, etc. In some examples, the apneas may comprise obstructive sleep apnea, central sleep apnea, etc.

In some examples, the tool-gripping portion 70 is closer to the distal tip 62 of the lead 50 than the proximal portion 140. In some examples, the tool-gripping portion 70 forms part of a distal portion 95 (FIG. 2B) of a lead, such as within 5 percent of the length of lead 50 as measured from the distal tip 62. In some such examples, the tool-gripping portion 70 is located within 10 centimeters of the distal tip 62 of the lead 50. In some such examples, a proximal end 79 of the tool-gripping portion 70 is located within 10 centimeters of the distal tip 62 of the lead 50. In some such examples, the first anchor 80 is located within 15 centimeters of the distal tip 62 of the lead 50.

In some examples, the tool-gripping portion 70 forms part of a distal portion 95 of a lead, such as within 10 percent of the length of lead 50 as measured from the distal tip 62. In some examples, the tool-gripping portion 70 forms part of a distal portion 95 of a lead, such as within 20 percent of the length of lead 50 as measured from the distal tip 62.

In some examples, the tool-gripping portion 70 forms part of a distal portion 95 of a lead, such as greater than 5 percent of the length of lead 50 as measured from the distal tip 62 and less than 10 percent of the length of lead 50 as measured from the distal tip 62.

FIG. 2B is a diagram 150 including an enlarged partial top plan view schematically representing a distal portion of an example lead 50, including at least first portion 60, tool-gripping portion 70, and anchor 80. As shown in FIG. 2B, in some examples first portion 60 comprises a non-gripping portion 66 including an operative element 64. In some such examples, the sensor portion 60 may comprise a proximal segment, which is not visible in FIGS. 2B-2C, but which is illustrated as element 210 in at least FIGS. 3, 7-8, 11-12. The indicator 63 designates a boundary or border between the tool-gripping portion 70 and the non-gripping portion 66 of the first portion 60. In some examples, the size and/or stiffness of the tool-gripping portion 70 between the anchor 80 and the non-gripping portion 66 can be selected during manufacturing. In some examples, implementing additional stiffness in the tool-gripping portion 70 may enhance the control of the non-gripping portion 66 when holding the tool-gripping portion 70. In some examples, implementing additional flexibility in the tool-gripping portion 70 may be beneficial to mechanically isolate physiologic motion between the anchor (e.g. 80) and the non-gripping portion 66 after implant.

In some examples, the electrically non-conductive outer portion 260 (FIG. 3) of the tool-gripping portion 70 is sufficiently flexible and yields an adequate amount to permit a firm grip to be established via a tool (or otherwise), thereby allowing the surgeon to control insertion as well as rotational orientation of the lead tip. Among other attributes, this example arrangement may reduce the potential for slipperiness, hindering establishment of a firm grip, in which an insufficiently flexible electrically non-conductive outer portion might otherwise hinder establishment of a firm grip.

In some examples, the operative element 64 may comprise a sensing element, which is contained within and/or at least partially exposed on the non-gripping portion 66. In some such examples, the sensing element may comprise a pressure-indicative sensor. In some examples, the operative element 64 may comprise a stimulation element, which is contained within and/or at least partially exposed on the non-gripping portion 66. In some examples, the stimulation element may comprise an electrode for placement in contact with a tissue to be stimulated. In some such examples of a stimulation element, the electrode may be at least partially exposed on a surface of the first portion 60.

As shown in FIG. 2B, first anchor 80 comprises a main portion 82, which includes a body 89 and a pair of arms 83 extending outwardly from opposite sides of the body 89. In some examples, each arm 83 may comprise a hole 84 for use in suturing the first anchor 80 relative to surrounding tissues and structures within the patient's body. In some examples, first anchor 80 comprises transition members 88 located on opposite ends of the main portion 82, with one transition member 88 comprising a distal end 81A of the entire first anchor 80 and the other transition member comprising a proximal end 81B of the entire first anchor 80.

In some examples, first anchor 80 may comprise an array 86 of topographic features 87 on main portion 82. The topographic features 87 may comprise protrusions and/or recesses, or combinations thereof. The topographic features 87 may be in one dimension (e.g. x), two dimensions (e.g. x and y), or even three dimensions (e.g. x, y, z) to aid visualization. In some examples, some topographic features 87 are located on the body 89 while some topographic features 87 are located on arms 83. In some examples, the topographic features 87 may form a pattern, such as a diamond pattern or other pattern recognizable via tactile senses of a user's thumb or finger and/or recognizable visually.

As further shown in the side view diagram 180 of FIG. 2C, in some examples the topographic features 87 may be located solely on a second surface 172 (e.g. top face surface) of the first anchor 80. In some such examples, the operative element 64 (of non-gripping portion 66 of first portion 60) is located on an opposite first surface 174 (e.g. bottom face) of the first portion 60 opposite the topographic features 87. Via this configuration, upon implantation the operative element 64 will face toward (e.g. downwardly in some examples) target tissues to facilitate the function of the operative element 64 (e.g. sensing, stimulation, other) while the topographic features 87 will face in an opposite direction (e.g. upwardly in some examples) to be sensed tactilely and/or visually by a surgeon during implantation (e.g. advancement, positioning) of the first portion 60. In some examples, the topographic features 87 are employed as visual indicators to indicate the relative orientation of the function element 84 even when the topographic features 87 on the first anchor 80 are not used via tactile sensing by a thumb or finger of the surgeon. As noted elsewhere, in some examples the operative element 84 may comprise electrical, mechanical, chemical, and/or thermal functions such that the orientation of the function element 84 relative to the target tissue may affect the role of the operative element 84. Accordingly, the topographic features 87 on the first anchor 80 may enhance proper placement of the operative element 84 in its desired orientation.

In some examples, the first anchor 80 comprises a third visual designator 85, which may comprise the same types or different types of visual designation as the first visual designator 75 of the tool-gripping portion 70. In some examples, the third visual designator 85 is different from the second visual designator 65 to help differentiate the first anchor 80 from the tool-gripping portion 70. In some examples, the third visual designator 85 may have the same appearance as the second visual designator 65.

In some examples, the third visual designator 85 may comprise a radiopaque component, such that the first anchor 80 is highly visible under radiologic imaging. Moreover, in some such examples, the radiopaque component may result in the first anchor 80 having a white, opaque appearance. In some such examples, this white opaque appearance stands in sharp contrast to the second visual designator 65 of the non-gripping portion 66, which may have a transparent, stainless steel appearance. The white opaque appearance of the third visual designator 85 also may stand in sharp contrast to the first visual designator 75 of the tool-gripping portion 70, which may be the color blue or another color which stands in sharp contrast to the environment within the patient's body (e.g. bodily fluids, tissues, blood, etc.), the first anchor 80 and the non-gripping portion 66.

In some examples, the first anchor 80 may be considered as a tool-grippable element, which may effectively extend the length of lead 50 which may be gripped by a tool during implantation.

As further shown in FIG. 2B, the tool-gripping portion 70 may comprise a primary gripping portion 142 and a transition portion 144 between the first anchor 80 and the primary gripping portion 144. Further details regarding such portions 142, 144 are further described later in association with at least FIGS. 3 and 6A-9D.

In some examples, second anchor 90 comprises at least some of substantially the same features and attributes as first anchor 80 in FIGS. 2B-2C.

FIG. 3 is a diagram 200 including an enlarged partial Feature Setal view of the tool-gripping portion 70 of an example implantable lead 50 (FIGS. 1-2C). In some examples, the tool-gripping portion 70 and other features shown in FIG. 3 may comprise at least some of substantially the same features and attributes as the tool-gripping portion 70 (and related features) as previously described in association with FIGS. 1-2C.

As shown in FIG. 3, the tool-gripping portion 70 and at least a proximal portion 210 of the first portion 60 are shown in Feature Set. In particular, first portion 60 comprises a distal portion 230 (forming non-gripping portion 66 in FIGS. 1-2C) and a proximal portion 210 extending proximally from the distal portion 230 with identifier 63 identifying a border between the respective portions 230, 210. In some examples, an outer surface 216 of the proximal portion 210 forms a shoulder 232 relative to the outer surface 231 of the distal portion 230 of the first portion 60.

As further shown in FIG. 3, the proximal portion 210 includes an inner wall structure 214, which defines a lumen 215 through which additional conductive elements (e.g. wires) may extend to connect conductive element(s) in the lead body 100 to the operative element 64 (FIGS. 2A-2C) in first portion 60.

As shown in FIG. 3, in some examples the tool-gripping portion 70 comprises an electrically non-conductive outer portion 260 and an electrically conductive inner portion 240. The electrically conductive inner portion 240 comprises an inner surface 281 and an opposite outer surface 283, and a distal end 284A which is connected electrically and mechanically relative to the proximal portion 210 of the sensor portion 60. In some examples, the electrically conductive inner portion 240 comprises an elongate tubular structure, which is flexible and resilient yet holds a generally linear shape until an external force induces a curved deflection in the shape along the length of the inner portion 240. In some examples, the electrically conductive inner portion 240 has an outer diameter D1, which is substantially less than an outer diameter D2 of the electrically non-conductive portion 260.

In some examples, the tool-gripping portion 70 may comprise a distal segment 440, a proximal segment 442, and a transition segment 444. In one aspect, a combination of the distal segment 440 and proximal segment 442 correspond to the primary portion 142 identified via dashed lines in FIG. 2B, while the transition segment 444 in FIG. 3 may correspond to the transition portion 144 identified via dashed lines in FIG. 2B.

In some examples, the thickness T1 of the electrically non-conductive outer portion 260 in the proximal segment 442 of tool-gripping portion 70 is substantially greater than the thickness T2 of the electrically non-conductive outer portion 263 in the distal segment 440 of the tool-gripping portion 70. In some examples, the thickness T1 of the proximal segment 442 of the tool-gripping portion 70 is at least one order of magnitude greater than the thickness T3 of an electrically non-conductive outer layer 270 of the non-gripping portion 66 of first portion 60, as shown in FIG. 4.

In some examples, at least in the region of the electrically non-conductive outer portion 260, tool-gripping portion 70 has an outer diameter D2, which in some examples is the same or substantially the same as the outer diameter D2 of the non-gripping portion 66 of the first portion 60. In some examples, the outer diameter D2 is constant or substantially constant and the outer diameter D2 of the non-gripping portion 66 (including distal tip 62) is constant or substantially constant. In some examples, the outer diameter D4 of the proximal portion 210 of the first portion 60 is less than the outer diameter D2 of the non-gripping portion 66 of the first portion 60. In some examples, the difference between diameters D2 and D4 generally corresponds to the thickness T2 of the distal segment 440 of the tool-gripping portion 70. Via this arrangement, the outer surface 269B of the distal segment 440 of the tool-gripping portion 70 sits generally flush with the outer surface 231 of the non-gripping portion 66 of the first portion 60.

In some examples, via this arrangement the lead 50 may comprise a constant outer diameter profile or near constant outer diameter profile of the first portion 60 and tool-gripping portion 70, which may facilitate ease of insertion and subcutaneous advancement into and through a subcutaneous pocket, as least as compared to an outer diameter profile which is highly variable such as having a greater outer diameter at a distal tip as in some commercially available devices, which can experience greater resistance during insertion and advancement.

In some examples, the electrically conductive inner portion 240 of tool-gripping portion 70 extends proximally through the length of the lead body 100 to its proximal end 131B to at least partially define the structure of the lead body 100. In some examples, the electrically conductive inner portion 240 may comprise a cable, coil, coiled cable or similar structure suitable for forming a lead body 100, as further described later in association with FIG. 13A.

As further shown in FIG. 3, at least the proximal segment 442 of the tool-gripping portion 70 comprises an electrically non-conductive outer portion 260 comprising an outer surface 264 and opposite inner surface 265, which is coaxially arranged about the outer surface 283 of the electrically conductive inner portion 240. In some examples, this proximal segment 442 has a length L9 and a thickness T1.

Meanwhile, a distal segment 440 of the tool-gripping portion 70 comprises an electrically non-conductive outer portion 263 having an outer surface 269B and opposite inner surface 269A, which is coaxially arranged about outer surface 216 of the proximal portion 210 of the first portion 60. In one aspect, the unexposed proximal portion 210 of the first portion 60 may be considered to be at least a portion of the electrically conductive inner portion underlying the electrically non-conductive outer portion 260 of the tool-gripping portion 70. In some examples, this unexposed proximal portion 210 may sometimes be referred to as an unexposed proximal segment, with the term unexposed referring at least to the proximal portion 210 of first portion 60 not being exposed to the environment but rather concealed via electrically non-conductive outer portion 263.

In some examples, an outer surface 216 of the proximal portion 210 forms a shoulder 232 relative to the outer surface 231 of the distal portion 230 of the first portion 60.

Via this arrangement, the distal segment 440 of the tool-gripping portion 70 overlaps, and is coextensive with, the proximal portion 210 of the first portion 60. In one aspect, this proximal portion 210 of first portion 60 at least partially acts as a structural support for the tool-gripping portion 70. In some examples, this co-extensive and overlapping arrangement at least partially provides a robust mechanical coupling of the non-gripping portion 66 and tool-gripping portion 70 relative to each other such that continuity is exhibited in the lead in this region.

In addition, while not shown in FIG. 3 for illustrative clarity, the electrically conductive inner portion 240 may extend into, and within, an interior 215 of the proximal portion 210 of first portion 60, as at least partially illustrated in FIG. 12. The electrically conductive inner portion 240 being mechanically and electrically connected to interior conductive elements within interior 215 of proximal portion 210.

Via these example coupling arrangements, the non-gripping portion 66 and tool-gripping portion 70 may bend together as one and may avoid a hinge-like behavior at region of their overlap. In doing so, this arrangement may prevent or minimize one portion of the lead (e.g. non-gripping portion 66) exhibiting a sharp angle relative to another portion of the lead (e.g. first tool-gripping portion 70). Stated differently this co-extensive and overlapping arrangement may minimize or eliminate any potential “jack-knifing” behavior of the lead body of the tool-gripping portion 70 and the non-gripping portion 66 relative to each other.

In some examples, this distal segment 440 has a length L10, which may be less than the length L9 of the proximal segment 442 of the tool-gripping portion 70. In some examples, the distal segment 440 has a thickness T2 which is substantially less than the thickness T1 of the proximal segment 442. In some examples, in at least this context, the term “substantially less” corresponds to a difference of 25%, 50%, or 75%.

As further shown in FIG. 3, in some examples the tool-gripping portion 70 may comprise a transition segment 444, which is proximal to the proximal segment 442. The transition segment 444 is interposed between the first anchor 80 and the proximal segment 442 of the tool-gripping portion 70. The transition segment 444 has a length L11, which is substantially less than the length L9 of the proximal segment 442. In at least this context, the term “substantially less” corresponds to the transition segment 444 having a length L11 2×, 3×, 4× less than the length L9 of the proximal segment 442.

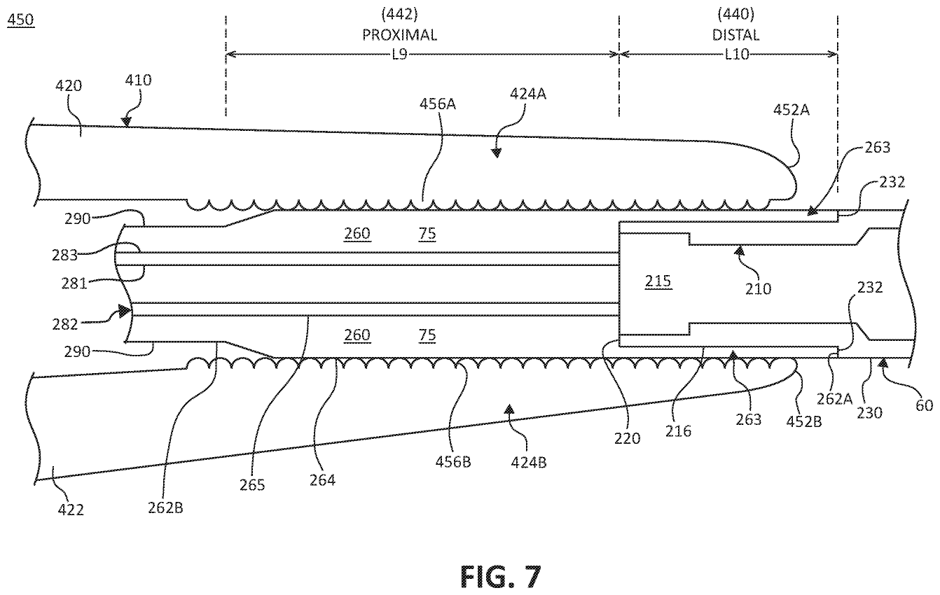

In some examples, the transition segment 444 of the tool-gripping portion 70 comprises an outer surface 290 which defines an outer diameter D5, which is less than the outer diameter D2 of the proximal segment 442 of the tool-gripping portion 70. In some such examples, the lesser diameter of the transition segment 444 may facilitate engagement of the proximal segment 442 and distal segment 440 of tool-gripping portion 70 by a tool, which may comprise tool 410 in FIGS. 6-7, tool 810 in FIGS. 14A-14C, tool 910 in FIGS. 15A-15C, or another tool.

FIG. 4 is an enlarged partial view of a portion of the outer surface 231 of the first portion 60, as identified via circle C, in one example implantable lead 50. As shown in FIG. 4, the first portion 60 comprises an electrically non-conductive coating or outer layer 270 which at least partially forms outer surface 231. Via this arrangement, the outer layer 270 provides electrical insulation from the surrounding environment within the patient's body for the underlying electrically conductive portions of the non-gripping portion 666 of first portion 60. In some examples, an outer surface 216 of the proximal segment 210 of first portion 60 also may comprise the electrically non-conductive coating 270.

In some examples, the electrically non-conductive outer portion 260 of tool-gripping portion 70 comprises a polymeric material, which is flexible, resilient and biocompatible. Accordingly, the outer portion 260 may flex and resume it's at rest state along with the flexible and resilient inner portion 240. The polymeric material has sufficient compression-resistance properties so as to compress minimally, such as when gripped via a tool, as further shown later in association with at least FIGS. 6A-9D.

In some examples, the electrically non-conductive outer portion 260 of tool-gripping portion 70 may comprise a second toughness, which is substantially greater than a first toughness of the electrically non-conductive outer layer 270 (FIG. 4) of the non-gripping portion 66 of first portion 60. In some examples, the second toughness is implemented via a combination of the type of material, the relative hardness of the material, and/or a thickness of the electrically non-conductive outer portion 260. Among other attributes, this second toughness is suited to significantly resist tearing, puncture, and the like.

Some example materials and construction of the electrically non-conductive outer portion 260 and the electrically conductive inner portion 240 are described later in association with at least FIGS. 12 and 13A-13B.

FIG. 5A is a diagram 300 including a front view schematically representing a patient's ribcage and example method of implanting a lead 350. As illustrated in FIG. 5A, a generator 355 is provided. In some examples, the generator 355 is implanted in a pectoral region of a patient in a manner known to the art such that generator 355 is positioned over a couple of ribs 304 of a ribcage 302. In some such examples, the generator 355 may sometimes be referred to as an implantable generator. In some such examples, the generator 355 may comprise a pulse generator to deliver stimulation pulses to stimulate a nerve and/or muscle, and/or the generator 355 may comprise a sensing generator to support sensing of physiologic information and/or environmental information.

With further reference to FIG. 5A, a lead 350 extends from the generator 355. In some examples, another lead (not shown) extends from generator 355 in a direction divergent from the lead 350. In some examples, lead 350 may be a sensor lead, stimulation lead, combined sensing-stimulation lead or other medical device 20 as described in association with at least FIG. 1.

In some examples, it will be understood that the portions of lead 350 proximal to first portion 360 have been implanted appropriately via tunneling and related known techniques prior to the implantation of the more distal portions of lead 350, such as but not limited to first portion 360, along with first and second anchors 380, 390.

As shown in method 300 in FIG. 5A, at least a serpentine segment 120 (FIG. 2A) of a lead body 352 extends down a lateral side of the rib cage 302 (and generally parallel to a longitudinal axis of the body) and first and second anchors 380, 380 are positioned to orient first portion 360 to extend generally parallel to ribs 304. In one aspect, a point 319 of entry is selected such that first portion 360 will become positioned between an adjacent pair of ribs.

In some examples, the first portion 360 comprises an operative element, such as operative element 64 in FIGS. 1-3. In some examples, the operative element 64 which comprises a sensing element such that first portion 360 may be considered a sensor portion. In some such examples method 300 includes implanting the sensor portion 360 over an adequate volume of lung tissue in order to obtain a representative measurement of respiratory effect and activity and/or a representative measurement of other respiratory-related information.

With this positioning shown in FIG. 5A, first anchor 380 (which is itself fixed relative to lead body 100) is secured in a fixed position at a lateral side of the body within the subcutaneous, extrapleural tissue region and lateral to the intercostal entry site 319 for the first portion 360. In some examples, second anchor 390 is secured relative to the lateral side of the body. In some examples, the second anchor 390 is permanently secured relative to the lead body 100, which may enhance long term stability and operation of the lead 350 within the patient's body.

FIG. 5B is a diagram that schematically illustrates the position of the lead 350 and first portion 360 relative to an incision area 321 and without illustrating the rib cage, as in FIG. 5A. As illustrated in FIG. 5B, the first and second anchors 380, 390 are located on the lateral side 303 of the patient's body to cause first portion 360 to be positioned lateral (toward outside of body) relative to a costochondral joint (represented by dashed lines 330) of nearby ribs. Moreover, the first anchor 380 is located and secured relative to body tissues external to the entry site 319 with the distal portion 356 of the lead extending through the intercostal entry site 319 to place the first portion 360 in the subcutaneous, extrapleural region between the adjacent ribs or other suitable location for interacting with target tissue, such as but not limited to sensing respiratory information.

It will be understood that an entry point for insertion of lead 350 and first portion 360 (into a desired position within the patient's body) may be other than that shown in FIGS. 5A-5B, such as the entry point being the same subcutaneous pocket in which a pulse generator (e.g. 355) is inserted and implanted. In other words, the insertion entry point need not be via a second, separate incision from the incision by which the pulse generator is implanted.

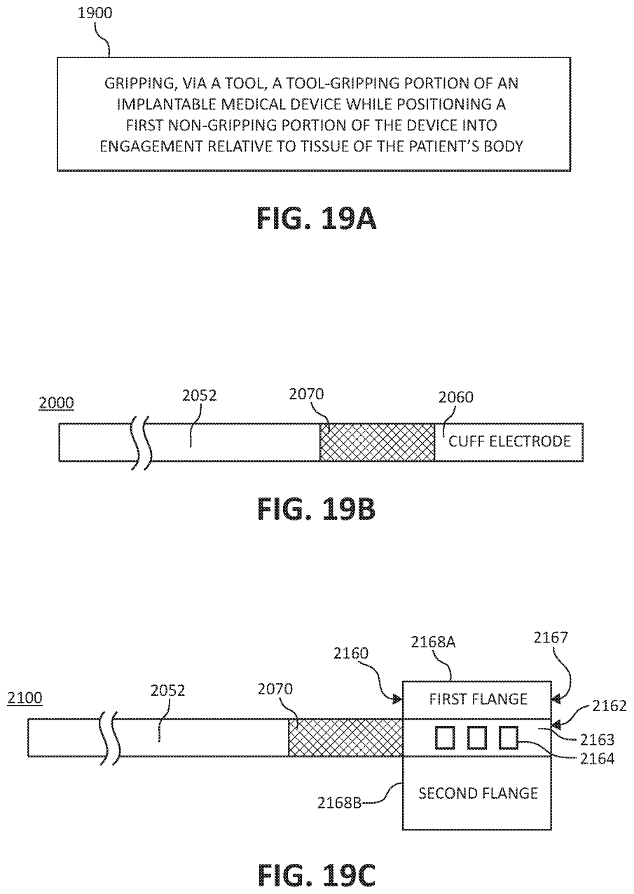

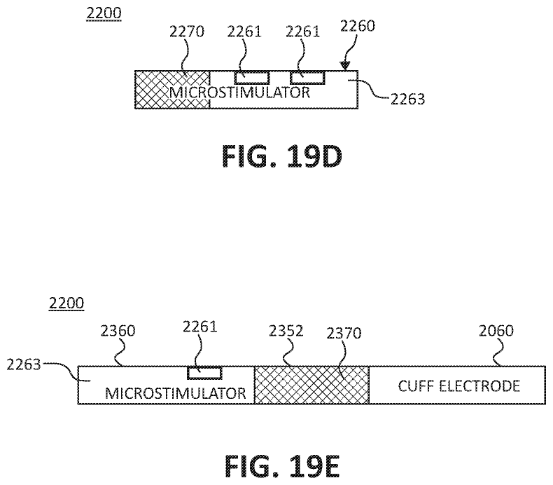

With regard to FIGS. 5A-5B, 6A-6C, 7-8, and 9A-9D, it will be understood that at least some aspects of such example methods may be performed via a lead (such as the previously described example leads) regardless of whether the operative element 64 of first portion 60 (or 360) comprises a sensing element, stimulation element or other type of operative element. Moreover, via the principles of such example methods, other example implantations of a first portion 60, 360 of a lead may be performed in other portions of the patient's body, different types of tissues, and/or for purposes other than obtaining respiratory information or treating respiratory issues. Moreover, with regard to FIGS. 5A-5B, 6A-6C, 7-8, and 9A-9D, it will be understood that at least some aspects of such example methods may be performed via a medical device (FIG. 1) other than a lead and in which an operative element 34 may comprise any one or more of the various types of modalities (e.g. electrical, mechanical, chemical, thermal, etc.) as previously described. With these general examples of the present disclosure in mind, some specific examples are described later in association with at least FIGS. 19A-19E.

FIGS. 6A-6C are a series of diagrams schematically representing an example implantation of an example implantable lead 450 in association with a tool-gripping portion 70 of the lead. In some examples, the implantable lead 450 comprises at least some of substantially the same features and attributes as the implantable lead(s), as previously described in association with at least FIGS. 1-5B. As shown in FIG. 6A, a tool 410 is used to grasp the tool-gripping portion 70 of the implantable lead 450. The tool 410 may comprise a proximal portion which has handles (not shown) or other means to control arms 420, 422 which may operate in a tongs-like fashion by which the two opposing arms 420, 422 may move toward and away from each other selectively. Movement of arms 420, 422 toward each other results in a gripping action on an object between the respective arms 420, 422. In this instance, the opposing arms 420, 422 act to grip the tool-gripping portion 70 in order to advance the first portion 360 of lead 450 subcutaneously beyond an incision site 419. The incision site 419 may comprise incision site 319 (FIGS. 5A-5B) or an incision site in a different portion of the patient's body and may relate to the same or different kind of tissues described in association with FIGS. 5A-5B.

As further shown in FIGS. 6A and 7, the tool 410 is positioned such that gripping portions 424A, 424B of arms 420, 422 are located just proximal to the border 63 between the tool-gripping portion 70 and the non-gripping portion 66 of the first portion 60. In this position, the gripping portions 424A, 424B of the arms 420, 422 releasably, securely engage both the distal and proximal segments 440, 442 of the tool-gripping portion 70 of lead 450 but do not engage transition segment 444 (FIG. 3) in some examples.

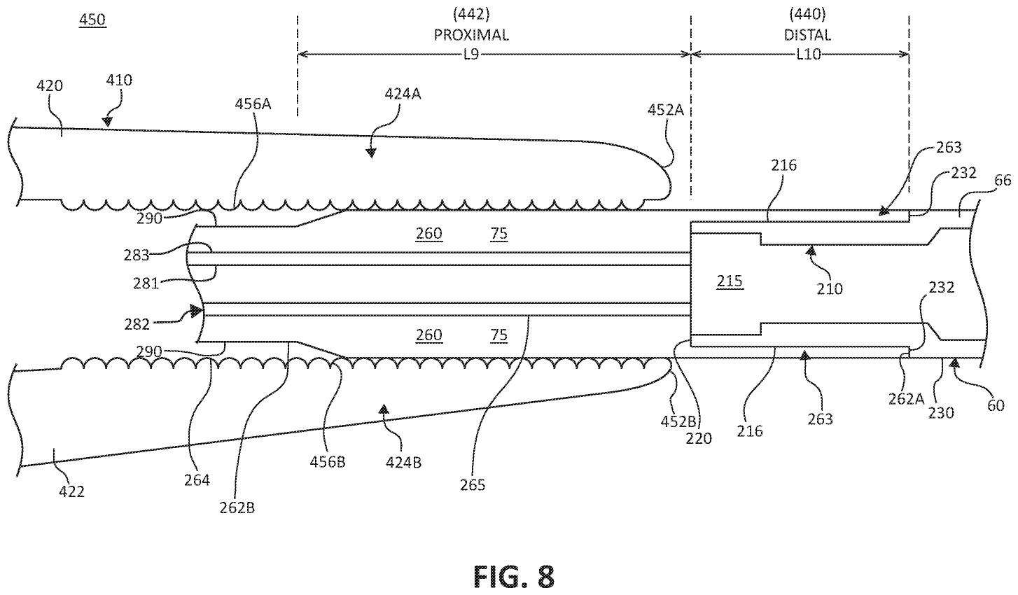

As shown in FIGS. 6A-6B, in some examples, the tool-gripping portion 70 may comprise a first visual designator 75 (e.g. FIGS. 2A-2C) which may facilitate locating and distinguishing the tool-gripping portion 70 from the first portion 60 and from the first anchor 80. As represented in FIGS. 7-8, the first visual designator 75 is not represented via a diamond pattern (e.g. FIGS. 1-4) for illustrative clarity but it will be understood that the reference numeral 75 in FIGS. 7-8 may comprises the same features and attributes as the first visual designator 75 shown and described in FIGS. 1-4.

Via this arrangement, the presence of the first visual designator 75 of tool-gripping portion 70 may deter placement and gripping of the arms 420, 422 on the non-gripping portion 66 of the first portion 60, thereby protecting the non-gripping portion 66, including the operative element 64 (FIGS. 2A-2C). Such deterrence includes protecting the relatively thin electrically non-conductive coating 270 (FIG. 4) overlying the electrically conductive outer surface 231 of the non-gripping portion 66 of first portion 60. Via such arrangements, the structure and location of the tool-gripping portion 70 of lead 450 may prevent unintentional or inadvertent penetration of the tool arms 420, 422 to electrically conductive elements of the lead body 450 and proximal segment 210 of the first portion 60.

With the tool 410 maintaining the gripped position on the tool-gripping portion 70 of lead 450 shown in FIG. 6A, the surgeon advances the non-gripping portion 66 through the incision site 419, such that the non-gripping portion 66 (shown in dashed lines) is no longer visible to the surgeon as the non-gripping portion 66 is further moved subcutaneously, as shown in the diagram 402 of FIG. 6B.

In the position shown in FIG. 6B, the tool-gripping portion 70 of lead 450 remains visible, as aided via the presence of first visual designator 75.

While still maintaining the same gripping position of tool 410 on tool-gripping portion 70 of lead 450, the non-gripping portion 66 of first portion 60 is advanced even further subcutaneously beyond the incision site 419. In FIG. 6C, the dashed lines represent the non-gripping portion 66 and the arms 420, 422 of the tool 410 in their gripped position on the tool-gripping portion 70 of lead 450, which is the same gripped position shown in FIGS. 6A and 6B. In some examples, the non-gripping portion 66 is advanced until the first anchor 80 becomes positioned just proximal to the incision site 419, as shown in FIG. 6C, and at which the first anchor 80 may be secured to thereby anchor the non-gripping portion 66 relative to the target area to be sensed and/or stimulated (or otherwise interacted with). Once the desired positioning is achieved, the surgeon may manipulate the tool 410 to release the grip on the tool-gripping portion 70 of the lead 50 and remove the tool 410 proximally from the subcutaneous pocket while leaving the non-gripping portion 66 (including operative segment 64) in its desired location.

Accordingly, because the tool-gripping portion 70 of lead 450 is configured to robustly withstand gripping pressure, the surgeon can still further advance the non-gripping portion 66 of lead 450 with confidence in the preserving the structural integrity of: the electrically non-conductive outer portion 260 of the tool-gripping portion 70; the electrically non-conductive coating 270 on the non-gripping portion 66 of first portion 60; and the underlying conductive structures in the non-gripping portion 66 of first portion 60.

In some instances, it may be desirable to obtain a gripping position on tool-gripping portion 70 of lead 450 in which the arms 420, 422 of tool 401 are located more proximally than shown in FIGS. 6A-6C, 7. A more proximal gripping position may enable further advancing the non-gripping portion 66 of first portion 60 within a subcutaneous pocket and/or may further ensure the confidence of the surgeon in preserving the structural integrity of the electrically non-conductive outer layer 270 on the non-gripping portion 66, the conductive structures of non-gripping portion 66 underlying outer layer 270, and/or of the electrically non-conductive outer layer 260 of the tool-gripping portion 70. In some instances, the more proximal gripping position may be implemented to overcome increased resistance which is sometimes encountered when subcutaneously advancing the non-gripping portion 66 of the first portion 60.

With this in mind, as shown in FIG. 8, in some example the arms 420, 422 of tool 410 may be positioned more proximally such that the arms 420, 422 releasably, securely engage the proximal segment 442 of the tool-gripping portion 70 of lead 50 without engaging (or minimally engaging) the distal segment 440 (FIG. 3) of the tool-gripping portion 70. In this arrangement, the distal tips 452A, 452B of arms 420, 422 of tool 410 are positioned proximal to the distal segment 440 of tool-gripping portion 70 and proximal to the proximal end 220 of the proximal segment 210 of first portion 60. Nevertheless, in this more proximal position, the gripping portions 424A, 424B of arms 420, 422 of tool 410 do not engage the transition segment 444 of tool-gripping portion 70.

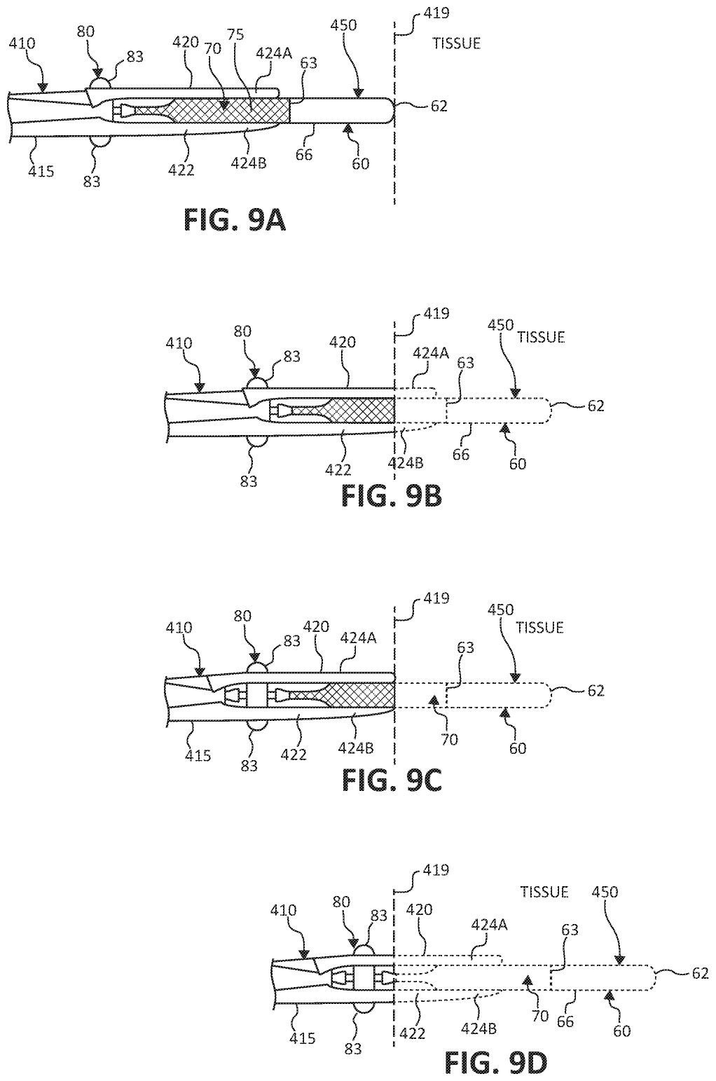

FIGS. 9A-9D are a series of diagrams schematically representing an example implantation of an example implantable lead in association with a tool 410 gripping a portion of the lead 450. As shown in FIG. 9A, a surgeon may use tool 410 to obtain a gripping position similar to that shown in FIG. 6A in which this gripping position is used to subcutaneously advance the non-gripping portion 66 of first portion 60 of lead 450 as shown in FIG. 9B. FIG. 9B depicts the non-gripping portion 66 of first portion 60 as having been advanced further subcutaneously than shown in FIG. 6B. However, in some examples, either because a deeper subcutaneous pocket is present or perhaps more resistance than usual is encountered, the surgeon may desire to establish a more proximal gripping position. Accordingly, the surgeon may release the tool 410 from the gripping position shown in FIG. 9B, and adopt a more proximal gripping position on the tool-gripping portion 70 of lead 450 as shown in FIG. 8 and FIG. 9C. With this more proximal gripping position, the surgeon further subcutaneously advances the non-gripping portion 66 of first portion 60 within the tissue until the non-gripping portion 66 of first portion 60 achieves the position shown in FIG. 9D. This position is represented by the dashed lines in FIG. 9D, which represent the non-gripping portion 66, tool-gripping portion 70, and arms 420, 422 of the tool 410. In a manner similar to that previously described in association with FIG. 6C, the subcutaneous advancement may be ceased when the first anchor 80 (which is fixed to the lead body 100) becomes positioned just proximal to the incision site 419. At this point, the surgeon may manipulate tool 410 to release arms 420, 422 from their grip on tool-gripping portion 70 and withdraw the tool 410 from the subcutaneous pocket while leaving the first portion 60 in its fully implanted position.

It will be understood that, in some examples, a surgeon may initially adopt the gripping position shown in FIG. 8 and FIGS. 9C-9D without having first adopted a gripping position like that shown in FIGS. 6A-6D, 7.

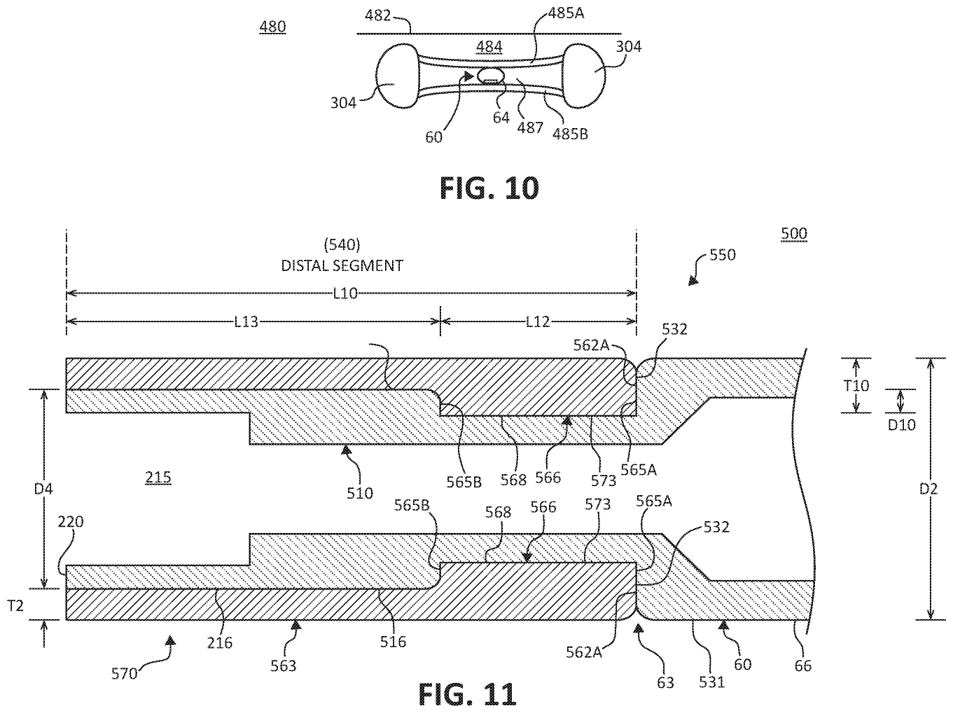

FIG. 10 is a diagram 480, including a side Feature Setal view, schematically representing an example implantation of a first portion (including an operative element 34, 64) of an example implantable medical device, such as a lead. As shown in FIG. 10, non-gripping portion 66 of first portion 60 is positioned in pocket 487 between adjacent, spaced apart ribs 304, and between layers 485A, 485B of tissue/muscle. In its implanted position, in some examples the operative element 64 faces downward toward the respiratory system and away from the external skin surface 482. In some such examples, the operative element 64 may comprise a sensing element, and in some examples, the operative element 64 senses respiratory-related information.

FIG. 11 is a diagram 500 including an enlarged partial Feature Setal view schematically representing a distal segment 540 of the tool-gripping portion 570 of an example implantable lead 550. In some examples, the lead 550 comprises at least some of substantially the same features and attributes as the leads as previously described in association with FIGS. 1-10, except having a modified distal segment 540 of the tool-gripping portion 570. In particular, as shown in FIG. 11, in some examples, a distal segment 540 of the tool-gripping portion 570 comprises an electrically non-conductive outer portion 563 including a protrusion 566 which has a size and shape to fit within a correspondingly sized and shaped recess 573 of an outer surface 516 of the proximal portion 510 of the first portion 560. The protrusion 566 comprises a main surface 568 extending between opposite end surfaces 565A, 565B. In the area of protrusion 566 of electrically non-conductive outer portion 563, the distal segment 540 has a thickness T10 which is substantially greater than a thickness T2 of more proximal portions of the distal segment 540, as shown in FIG. 11. In this context, the term “substantially greater” corresponds to a difference in thickness of about 20%, 30%, 40%, 50%, and the like. In some examples, the recess 573 has a depth D10, which corresponds to the amount of increased thickness of protrusion 566 relative to the remainder of the electrically non-conductive outer portion 563 of distal segment 540 of the tool-gripping portion 570. In one aspect, one end of the recess 573 may be considered as forming a shoulder 532 relative to outer surface 531 of non-gripping portion 566 of first portion 560.

In general terms, the combination of the protrusion 566 of the electrically non-conductive outer portion 563 and the recess 573 (within the outer surface of the proximal portion 510 of the first portion 510) act to securely retain the electrically non-conductive outer portion 563 of the distal segment 540 of tool-gripping portion 570 and prevent or minimize its movement longitudinally away from the first portion 560. In some examples, this movement is prevented or minimized during a curing process, such as might occur after backfill material is added to fill minor gaps between major components of the first portion 560 and the tool-gripping portion 570. Moreover, during assembly of the electrically non-conductive outer portion 563 relative to the proximal portion 210 of the first portion 560, the combination of the protrusion 566 and the recess 573 may act as a locating feature to facilitate proper longitudinal placement of the electrically non-conductive outer portion 563 of the tool-gripping portion 570 relative to the first portion 560.

In addition, in some examples, the increased thickness of the protrusion 566 of the electrically non-conductive outer portion 563 also may serve to increase the insulation and gripping thickness at a point just proximal to the border 63 between the non-gripping portion 566 of first portion 560 and the tool-gripping portion 570. This location is one in which the distal tips (e.g. 452A, 452B in FIG. 8) of a gripping tool 410 may be likely to apply forces on the tool-gripping portion 570 during implantation of lead 550. It will be understood that the protrusion 566 may have a size and/or shape other than that shown in FIG. 11, with recess 573 also having a different size and/or shape suited to correspond to the differently sized and/or shaped protrusion 566.

FIG. 12 is an enlarged partial Feature Setal view schematically representing at least some components of a tool-gripping portion 670 of an example implantable lead 600. In some examples, lead 600 comprises at least some of substantially the same features and attributes as the leads (and their tool-gripping portion 70, 570) as previously described in association with FIGS. 1-11, except with the lead 600 in FIG. 12 providing a particular manner of constructing the electrically non-conductive outer portion 640 of the tool-gripping portion 670.

FIG. 12 provides just one example implementation by which the electrically non-conductive outer portion 660 and/or electrically conductive inner portion 640 of the tool-gripping portion 608 may be implemented.

As shown in FIG. 12, in some examples the electrically non-conductive outer portion 660 of the tool-gripping portion 670 comprises a first sleeve 608, a second sleeve or tube 630, and a transition member 671. Accordingly, each of the elements 608, 630, 671 is made of an electrically non-conductive material, which can be the same material or different materials for each element 608, 630, 671. In each instance, the electrically non-conductive material comprises a flexible, resilient material. In some examples, each element 608, 630, 671 forms an elongate annular, cylindrical-shaped member, which may sometimes be referred to as being tubular.

In some examples, the electrically conductive inner portion 640 comprises an elongate structure(s) suitable to conduct electrical power and/or signals between a proximal end and distal end of the lead body, with such elongate structure(s) able to take a wide variety of forms, shapes, sizes, and materials. Just a distal portion of the electrically conductive inner portion 640 is shown in FIG. 12 for illustrative simplicity. In some examples, the electrically conductive inner portion 640 comprises an outer surface 683 and may comprise an inner surface 681 in some examples.

In some such examples, the electrically conductive inner portion 640 comprises at least two different conductive elements which independently conduct power and/or signals from each other. One example implementation of the electrically conductive inner portion 640 is described later in further detail in association with FIG. 13A. In some examples, the distal end 647 of the electrically conductive inner portion 640 protrudes within lumen 215 of the proximal portion 210 of the first portion 260 to facilitate mechanical anchoring and electrical connection to components within the proximal portion 210 of the first portion 260. As previously mentioned, this robust mechanical coupling may prevent any potential “jack-knifing” of the tool-gripping portion 670 relative to the first portion 60 during implantation of the lead or other internally-deliverable medical device.

With this in mind, the elements 608, 630, 671 comprising electrically non-conductive outer portion 660 act together to form an electrically non-conductive barrier to prevent exposure of the electrically conductive inner portion 640 to the environment within the patient's body.

In some examples, the second sleeve 630 extends between a distal end 632A and proximal end 632B, and has a length (L15). As shown in FIG. 12, in some examples, the distal end 632A of the second sleeve 630 is spaced apart from the proximal end 220 of the proximal portion 210 of the first portion 260, with tubular member 671 interposed between the second sleeve 630 and the proximal portion 210 of the first portion 260.

The transition member 671 has opposite ends 672A, 672B. In some examples, transition member 671 is formed via backfilling a planned gap among the other components (e.g. sleeve 608, 630, etc.). However, in some examples transition member 671 may comprise a sleeve. In some examples, the transition member 671 comprises approximately the same thickness as the second sleeve 630 and has a length (L17) filling the space between the second sleeve 630 and the proximal portion 210 of the sensor portion 60.

In some examples, the electrically non-conductive outer portion 660 comprises a third sleeve 650 which coaxially extends about the electrically conductive inner portion 640, and which is interposed between the electrically conductive inner portion 640 and the second sleeve 630. In some such examples, the third sleeve 650 extends proximally along substantially the entire length of the lead body (e.g. 100 in FIG. 2A) to electrically and mechanically protect the electrically conductive inner portion 640 from the environment within the patient's body.

In some examples, the first and second anchors 80, 90 (e.g. FIGS. 2A-2C) are coaxially disposed over a sleeve such as the third sleeve 650, and in a fixed position along the length of the third sleeve 650 (and therefore the length of the lead body 100 in FIG. 2A). In some examples, the lead 600 may further comprise a mounting sleeve (not shown), which is coaxially mounted about the third sleeve 650 and which has a length generally corresponding to a length of each respective first or second anchor 80, 90.

In some examples, the electrically conductive inner portion 640 comprises a conductive structure, which can take a wide variety of forms with one example conductive structure 740 comprising a coil-shaped structure, such as shown in the example of FIG. 13A. In some such examples, the coil-shaped conductive structure 740 may comprise a co-radially arranged pair of conductors 752, 753 in which the conductors 752, 753 may comprise an electrically non-conductive coating or outer layer 760 covering a conductive core 756, as shown in FIG. 13B. In some such examples, the coil-shaped conductive structure 740 forms just one part of a coil-shaped conductive structure extending throughout substantially the entire length of the lead body (e.g. 100 in FIG. 2A). In some examples, the conductive structure 740 may comprise a pair of conductors arranged co-axially. It will be understood that in some examples, more than two conductors may form the lead body and as such the conductive structure 740 may take other forms and configurations by which a plurality of conductors form a flexible, resilient lead body, including a portion extending into, and forming an electrically conductive inner portion 640 of the tool-gripping portion 70.

In some examples, the electrically non-conductive outer layer 760 shown in FIG. 13B comprises a polymer material. In some such examples, this material comprises an ethylene tetrafluoroethylene (ETFE) material or similar material. The material is selected for its relative toughness and durability. In some examples, the outer layer 760 comprises a thickness T11 between about 0.0002 inches and about 0.01 inches. In some examples, this coating or outer layer 760 may be formed via extrusion during manufacturing of the individual conductors (e.g. wires, cables, strands, etc.). In some examples, the outer layer 760 of one of the conductors 752, 753 may be tinted with a color, e.g. blue in one example, to help visual identification of the lead body during implantation, handling, etc.

As further shown in FIG. 13A, in some such examples, the coil-shaped conductive structure 740 forms a wall 770 and which defines a lumen 772, with the wall 770 corresponding to the wall 645 in FIG. 12 and the lumen 772 corresponding to the lumen 648 in FIG. 12. Via the coil-shaped conductive structure 740, the electrically conductive inner portion 640 exhibits flexibility to permit bending the lead body during implantation and/or after implantation, as well as exhibiting resilience to return to a shape (e.g. straight or serpentine) to which the lead body is biased.

FIG. 14A is an isometric view schematically representing an example tool 810 for use in an example method of implanting a medical device. In some examples, tool 810 comprises at least some of substantially the same features and attributes as tool 410 as previously described in association with at least FIGS. 7-8.

In some examples, as shown in FIG. 14A, tool 810 comprises a pair of arm 820, 822 which comprise opposed gripping portions 824A, 824B. In some examples, the tool 810 comprises a pivot mechanism 833 to permit selective movement of the opposed gripping portions 824A, 824B toward each other for gripping the tool-gripping portion (e.g. 70 in FIG. 2A or 14B) and away from each other for releasing the gripping on the tool-gripping portion (e.g. 70) or to open the opposed gripping portions 824A, 824B to permit positioning the opposed gripping portions 824A, 824B of the tool 810 relative to the tool-gripping portion 70.

In some examples, such movement is controllable via opposed arms 872A, 872B, which each include a handle portion 874A, 874B. Via pivot mechanism 833, movement of the arms 872A, 872B causes a corresponding movement of arms 820, 822 and their respective gripping portions 824A, 824B.

As shown in FIG. 14B, in some examples the opposed gripping portions 824A, 824B are sized and shaped to generally correspond to a size and shape of the tool-gripping portion 70 of a lead 50. Via this arrangement, in one aspect the opposed gripping portions 824A, 824B more evenly distribute the gripping pressure over a larger surface area of the tool-gripping portion 70 than at least some commercially available gripping tools, which in turn may help protect the structural integrity of tool-gripping portion 70, including protection of electrically non-conductive outer portions or coatings.

In some examples, the opposed gripping portions 824A, 824B may comprise a smooth surface which may minimize concentrated loads, may comprise a surface of small protrusions and/or recesses to improve gripping action, or may comprise combination of smooth surfaces with at least some protrusions (and/or recesses).

In some examples, opposed gripping portions 824A, 824B each have a length (L20) which is at least one-half a length (e.g. L2 in FIGS. 2A-2B) of the tool-gripping portion 70. In some examples, the length L20 is at least three-quarters the length (L2) of the tool-gripping portion 70.

As further shown in FIG. 14C, in some examples, the opposed gripping portions 824A, 824B of the tool 810 are arranged to include a second arcuate cross-Feature Setal shape (e.g. arcuate or semi-circular) such that when joined together the overall shape generally corresponds to a first arcuate cross-Feature Setal shape (e.g. circular) of an outer surface of the tool-gripping portion (e.g. 70) of the implantable lead. As shown in FIG. 14C, the tool-gripping portion 824A comprises a pair of elongate contact edges 825A, 825B sized and shaped to releasably engage a pair elongate contact edges 827A, 827B having a corresponding size and shape. When contact edges 827A, 827B releasably engage contact edges 825A, 825B, respectively, then the opposed gripping portions 824A, 824B form a circular-shaped surface which releasably engages the generally circular outer surface 73 of tool-gripping portion 70 of lead 50. It will be understood that in some instances, even when the contact edges 827A, 827B do not completely releasably engage the contact edges 827A, 827B, the inner surfaces 821A, 821B of opposed gripping portions 824A, 824B may still releasably engage the outer surface 73 of tool-gripping portion 70 of lead 50 in a manner to exert sufficient gripping pressure to facilitate maneuvering the distal portions (e.g. sensor 60) of the lead 50 via tool 810.

After establishing the releasable engagement with the tool-gripping portion 70 of an implantable lead 50 as described above in association with FIGS. 14A-14C, the tool 910 may be used to push or otherwise maneuver the non-gripping portion 66 (including sensor portion 60) of thee lead 50 subcutaneously into a desired position, such as shown in FIGS. 6A-6C, FIGS. 9A-9D, and/or other implantation methods.

In some examples, the tool 810 is configured with a shape and/or cut-outs to accommodate the presence of anchors 80 and/or 90 (FIG. 2A).

FIG. 15A is an isometric view schematically representing an example tool 910 for use in an example method of implanting a medical device. In some examples, tool 910 comprises at least some of substantially the same features and attributes as at least tool 410, as previously described in association with at least FIGS. 6-7,

As shown in FIG. 15A, in some examples tool 910 comprises an operative portion 920 at a distal end 971A of a shaft 970, and a handle 980 at a proximal end 971B of shaft 970. As shown in FIGS. 15A-15B, the operative portion 920 comprises opposed gripping portions 924A, 924B arranged as elongate elements which are spaced apart from each other in a generally parallel relationship and spaced by a first distance D2 which is approximately the same as an outer cross-Feature Setal dimension (e.g. D2 in FIG. 3) of the tool-gripping portion 70. In some examples, the elongate elements are also generally perpendicular to the shaft 950 and may be perpendicular to handle 980. In some examples, the opposed gripping portion 924A, 924B have a circular or other arcuately-shaped cross-Feature Setal shape and may comprise rounded tips 927A, 927B. In some examples, the opposed gripping portions 924A, 924B are joined via a base portion 942C, which is connected to the distal end 971A of shaft 970.

As further shown in FIG. 15B, one example method comprises using tool 910 to grasp a tool-gripping portion 70 of the implantable lead. To do so, via handle 980 the elongate gripping portions 924A, 924B are positioned to be transverse to a longitudinal axis of the tool-gripping portion 70 and are positioned to slide over and onto a portion of the lead 50 until the tool-gripping portion 70 of the lead 50 becomes interposed between the opposed gripping portions 924A, 924B. In some such examples, a surgeon may apply some tension on the lead body of the implantable lead 50 in at least a region near handle 980 of tool 910 to facilitate maneuvering the opposed gripping portions 924A, 924B onto, and in releasable engagement with, the tool-gripping portion 70 of the implantable lead 50.

Via this arrangement, the tool 910 causes a friction fit engagement of the respective elongate gripping portions 924A, 924B against an outer surface 73 of the tool-gripping portion 70 of the implantable lead 50. After establishing this releasable engagement, the tool 910 may be used to push or otherwise maneuver the non-gripping portion 66 (including sensor portion 60) subcutaneously into a desired position, such as shown in FIGS. 6A-6C, FIGS. 9A-9D, and/or other implantation methods.

In some examples, commercially available “off-the-shelf” tools may be used to releasable engage (e.g. grasp) the tool-gripping portion 70 of an example implantable lead 50 and perform the example methods described herein to advance a sensor portion 60 of the lead 50 within a patient's body.

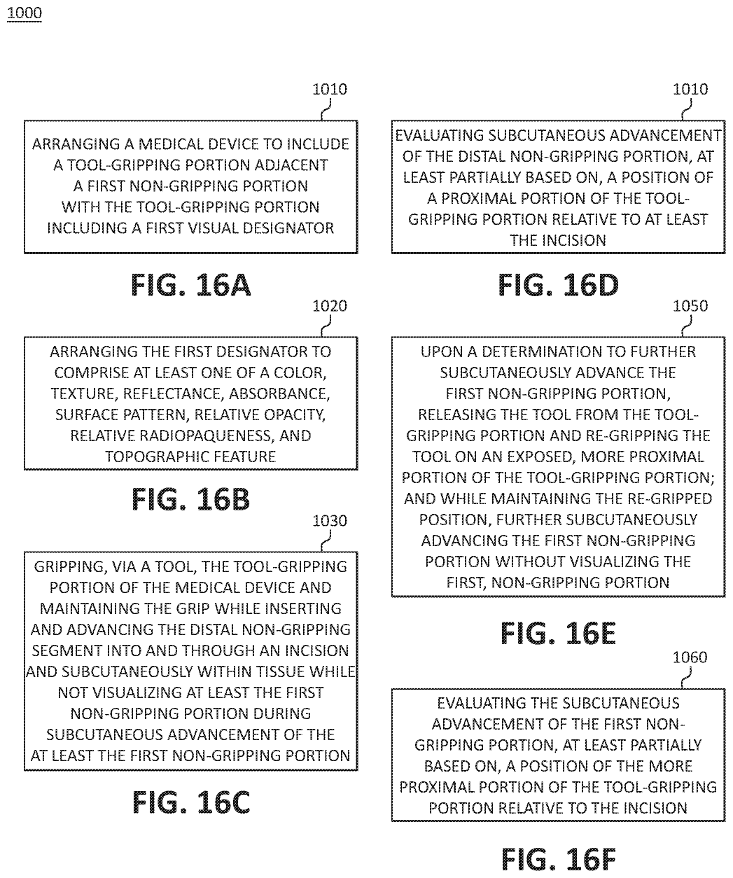

FIG. 16A is a flow diagram schematically representing an example method 1000. In some examples, method 1000 may be performed via at least some of substantially the same medical devices, leads, tool-gripping portions, anchors, tools, etc. as previously described in association with at least FIGS. 1-15C. In some examples, method 1000 may be performed via medical devices, leads, catheters, tool-gripping portions, anchors, tools, etc. other than those previously described in association with at least FIGS. 1-15C. As shown at 1010 in FIG. 16A, method 1000 comprises arranging a medical device to include a tool-gripping portion adjacent the non-gripping portion with the tool-gripping portion including a first visual designator. In some examples, the non-gripping portion may comprise a distal portion with the tool-gripping portion extending proximally adjacent the non-gripping portion.

As shown at 1020 in FIG. 16B, in some examples method 1000 in FIG. 16A may further comprise arranging the first visual designator to comprise at least one of a color, texture, reflectance, absorbance, surface pattern, relative opacity, relative radiopaqueness, topographic feature, and/or profile. In some examples, method 1000 may further comprising arranging a second visual designator, associated with the non-gripping portion, to be visually perceptibly different than the first visual designator. In some examples, method 1000 may comprise arranging the second visual designator to comprise at least one of a color, texture, reflectance, absorbance, surface pattern, relative opacity, relative radiopaqueness, topographic feature, and/or profile.

As shown at 1030 in FIG. 16C, in some examples method 1000 in at least FIG. 16A and/or FIG. 16B may comprise gripping, via a tool, the tool-gripping portion of the medical device and maintaining the grip while inserting and advancing the non-gripping portion of the lead into and through an incision and subcutaneously within tissue while not visualizing at least the non-gripping portion during subcutaneous advancement of at least the non-gripping portion (which may include an operative segment).

As shown at 1040 in FIG. 16D, in some examples method 1000 in at least FIGS. 16A and 16C may further comprise evaluating subcutaneous advancement of the non-gripping portion, at least partially based on, a position of a proximal portion of the tool-gripping portion relative to at least the incision.

As shown at 1050 in FIG. 16E, in some examples method 1000 in at least FIGS. 16A and 16D may further comprise that upon a determination to further distally, subcutaneously advance the non-gripping portion, releasing the tool from the tool-gripping portion and re-gripping the tool on an exposed, more proximal portion of the tool-gripping portion, and then while maintaining the re-gripped position, further subcutaneously advancing the non-gripping portion without visualizing the non-gripping portion.

As shown at 1060 in FIG. 16F, in some examples method 1000 in at least FIGS. 16A and 16E may further comprise evaluating the subcutaneous advancement of the non-gripping portion, at least partially based on, a position of the more proximal portion of the tool-gripping portion relative to the incision.

In some examples, the medical device implanted via the method(s) described in association with FIGS. 16A-16F may be implemented via implanting a lead, such as an implantable sensor lead, stimulation lead, or combination sensor-stimulation lead.

It will be understood that in some examples the same method can be performed except involving a catheter including non-gripping portion and tool-gripping portion adjacent the non-gripping portion, in which the non-gripping portion includes an operative element, such as but not limited to, drug delivery.

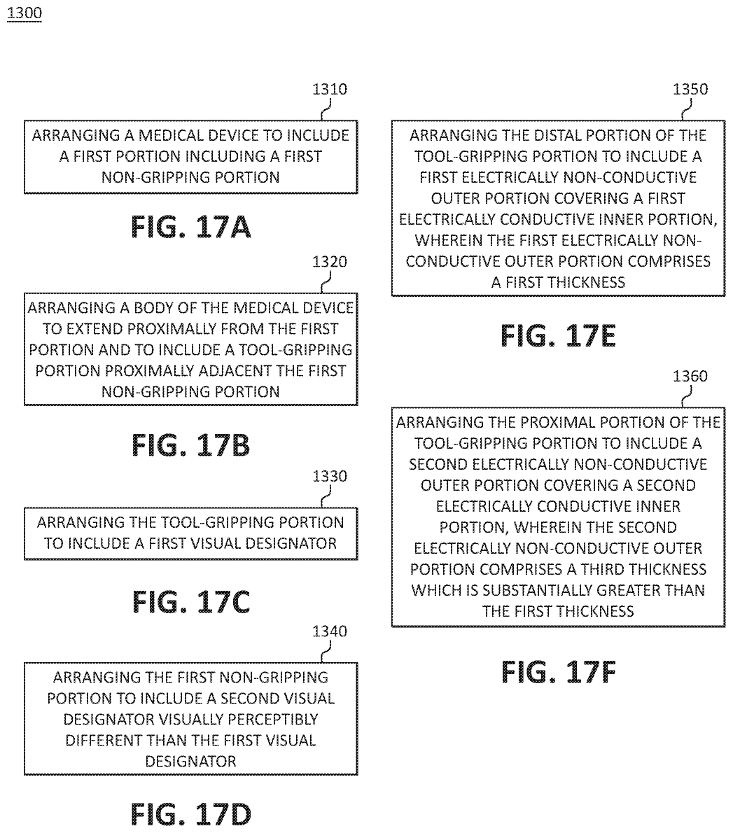

FIG. 17A is a flow diagram schematically representing an example method 1300. In some examples, method 1300 may be performed via at least some of substantially the same medical devices, leads, tool-gripping portions, anchors, tools, etc. as previously described in association with at least FIGS. 1-16F. In some examples, method 1300 may be performed via medical devices, leads, catheters, tool-gripping portions, anchors, tools, etc. other than those previously described in association with at least FIGS. 1-16F. As shown at 1310 in FIG. 17A, method 1300 comprises arranging a medical device to include a first portion including a non-gripping portion.

As shown at 1320 in FIG. 17B, in some examples method 1300 in FIG. 17A may further comprise arranging a body of the medical device to extend proximally from the first portion, and including a tool-gripping portion proximally adjacent the non-gripping portion.

As shown at 1330 in FIG. 17C, in some examples method 1300 in at least FIG. 17A may further comprise arranging the tool-gripping portion to include a first visual designator.

As shown at 1340 in FIG. 17D, in some examples method 1300 in at least FIG. 17A may further comprise arranging the non-gripping portion to include a second visual designator visually perceptibly different from the first visual designator.

As shown at 1350 in FIG. 17E, in some examples method 1300 in at least FIG. 17A may further comprise arranging the distal portion of the tool-gripping portion to include a first electrically non-conductive outer portion covering a first electrically conductive inner portion, wherein the first electrically non-conductive outer portion comprises a first thickness.

As shown at 1360 in FIG. 17F, in some examples method 1300 in at least FIG. 17A may further comprise arranging the proximal portion of the tool-gripping portion to include a second electrically non-conductive outer portion covering a second electrically conductive inner portion. In some examples, the second electrically non-conductive outer portion comprises a third thickness which is substantially greater than a diameter of the second non-conductive inner portion of the proximal portion of the visually designated tool-gripping portion.

It will be understood that at least some of the various example methods described in association with FIGS. 16A-16F and FIGS. 17A-17F may be performed together in different combinations in a complementary manner.

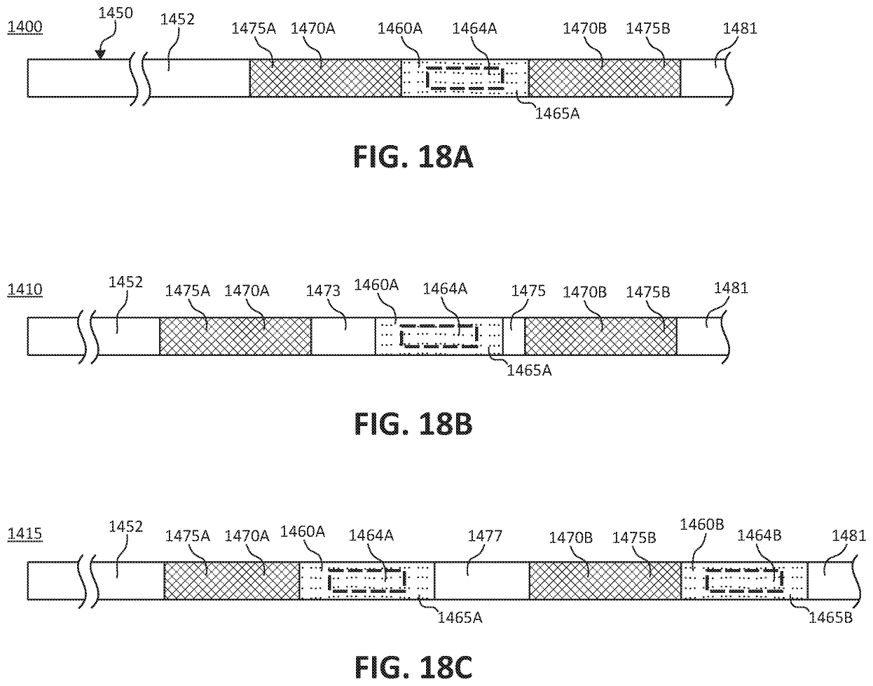

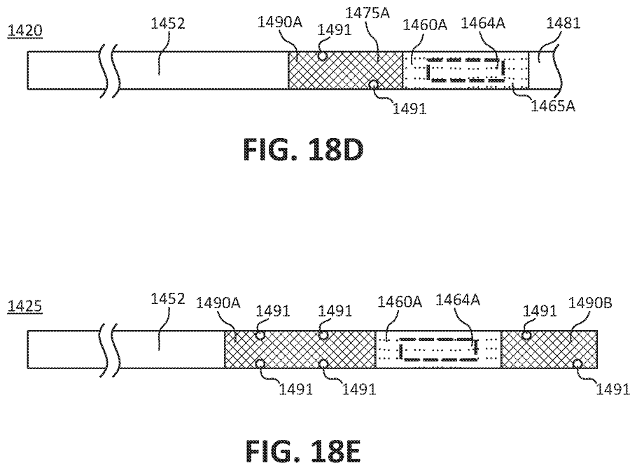

FIG. 18A is a top plan view schematically representing an example implantable lead 1400. In some examples, lead 1400 may comprise at least some of substantially the same features and attributes as the leads, medical devices, tool-gripping portions, non-gripping portions, anchors, etc. as previously described in association with FIGS. 1-17F. As shown in FIG. 18A, lead 1400 comprises at least two tool-gripping portions 1470A, 1470B spaced apart along lead body 1450 and with non-gripping portion 1460A interposed therebetween. In some examples, at least one of the respective tool-gripping portions 1470A, 1470B may comprise a visual designator 1475A, 1475B, like first visual designator 75 (FIGS. 2A-2C). The particular expression (e.g. color, texture, etc.) of visual designator 1475A may the same as or different from the particular expression (e.g. color, texture, etc.) of visual designator 1475B.

In some examples, the non-gripping portion 1460A may comprise operative element 1464A and may comprise a visual designator 1465A, like visual designator 65 in at least FIGS. 2A-2C.

In some examples of lead 1400, the tool-gripping portion 1470B may define a distal end of lead 1400 such that portion 1481 shown in FIG. 18A is absent or formed as a rounded tip (or other suitable shape). However, in some examples in which tool-gripping portion 1470B may be at any one of many possible locations along a length of lead 1410, then portion 1481 may represent a continuation of lead body 1452 other than a distal end of lead 1410.

FIG. 18B is top plan view schematically representing an example implantable lead 1410, which has substantially the same features and attributes as lead 1400 in FIG. 18A, except with the respective tool-gripping portions 1470A and/or 1470B being spaced apart from (e.g. not immediately adjacent or bordering) the non-gripping portion 1460A. In this example arrangement, lead body portion 1473 is interposed between tool-gripping portion 1470A and non-gripping portion 1460A, and lead body portion 1475 is interposed between tool-gripping portion 1470B and non-gripping portion 1460A.

FIG. 18C is top plan view schematically representing an example implantable lead 1415, which has at least some of substantially the same features and attributes as lead 1400 in FIG. 18B, except at least additionally comprising a second non-gripping portion 1460B, which may comprise an operative element 1464B. In some examples of lead 1415, the non-gripping portion 1460B may define a distal end of lead 1420 such that portion 1481 shown in FIG. 18C is absent or formed as a rounded tip (or other suitable shape). However, in some examples in which non-gripping portion 1460B may be at any one of many possible locations along a length of lead 1415, then portion 1481 may represent a continuation of lead body 1452 other than a distal end of lead 1415. In some examples, a lead body portion 1477 may be interposed between non-gripping portion 1460A and tool-gripping portion 1470B.

FIG. 18D is top plan view schematically representing an example implantable lead 1420. In some examples, lead 1420 comprises at least some of substantially the same features and attributes as the example medical devices previously described in association with at least FIGS. 1-17F, except comprising a tool-gripping portion 1490A which incorporates anchor elements 1491. As such, element 1490A is both a tool-gripping portion and an anchor. In some examples, the anchor elements 1491 comprise apertures formed within the lead body 1491 and/or formed within an outer sleeve fit over an external surface of the lead body 1491. When formed as part of the lead body 1452, reinforced sidewalls of lead body 1452 may comprise apertures which at least partially define the anchor elements 1491. In some such examples, the tool-gripping portion 1490A and the anchor (side wall and elements 1491) may be considered as a single or monolithic element having both a tool-gripping structure and an anchoring structure.

In some such examples, the tool-gripping portion 1490A also comprises a visual indicator 1475A, similar to first visual indicator 45 in FIG. 1, 75 in FIGS. 2A-2C.

In some examples of lead 1420, the non-gripping portion 1460A may define a distal end of lead 1420 such that portion 1481, which is shown in FIG. 18D, is absent or formed as a rounded tip (or other suitable shape).

In some examples, the anchor elements 1491 are located within a profile of the lead body 1452, i.e. they do not protrude outwardly from a general profile (e.g. outer surface/dimension) of the lead body 1452. In some examples, the anchor elements 1491 may protrude partially from a general outer surface of lead body 1452, such that the anchor elements 1491 are at least partially within an interior of the lead body 1452 such as shown in FIG. 18D.

Such example arrangements may facilitate tunneling the lead body 1452 subcutaneously at least because the outer profile of the lead body 1452 in the area of the anchor elements 1491 does not protrude or significantly protrude laterally outward from an outer wall of the lead body 1452, whereby such arms or protrusions which might otherwise hinder such tunneling. In some examples, incorporation of anchor elements with the tool-gripping portion also may facilitate insertion of the distal end of the lead (e.g. 50 in FIGS. 1-2C) via an introducer. For instance, the integrated anchor-tool-gripping portion 1490A could be used to push the lead 1420 through the introducer.

FIG. 18E is top plan view schematically representing an example implantable lead 1425. In some examples, lead 1425 comprises at least some of substantially the same features and attributes as the example medical devices previously described in association with at least FIGS. 1-17F, except comprising at least two tool-gripping portions 1490A, 1490B, each of which incorporates anchor elements. The two tool-gripping portions 1490A, 1490B are located on opposite ends of the non-gripping portion 1460A (which may comprise an operative element 1464A), which is interposed between the tool-gripping portions 1490A, 1490B. In some examples, one tool-gripping portion (e.g. 1490B) may be used to pull (or other manipulate) the lead body 1452 during gross movement in tunneling and/or subcutaneous positioning of the lead body 1452 and of the non-gripping portion 1460A. Meanwhile, in some examples, the other tool-gripping portion (e.g. 1490A) may be used for more fine movements in positioning of the lead body 1452 (and of non-gripping portion 1460A). However, either tool-gripping portion 1490A, 1490B can be used to facilitate gross and/or fine movements of the lead body 1452 in general and/or the non-gripping portion 1460A, and either (or both) tool-gripping portions 1490A, 1490B can be used for pushing and/or pulling as desirable. Moreover, as in the example arrangement of FIG. 18D, one or both of tool-gripping portions 1490A, 1490B in the example of FIG. 18E may comprise anchor elements 1491 having features and attributes as previously described.

While FIG. 18E depicts each respective tool-gripping portion 1490A, 1490B as incorporating anchor elements 1491, in some examples one or both of the tool-gripping portions 1490A may omit all or some of the anchor elements 1490A, 1490B.

With reference to FIGS. 18A-18E, it will be understood that in some examples, the tool-gripping portions (including or excluding anchor elements) may be distributed at different locations along different portions along the length of the lead body 1452. In some instances, one or more tool-gripping portions may be located at a distal end or proximal end of the lead body 1452 and/or may be located somewhere between the opposite ends of the lead body 1452.

As schematically represented via at least FIGS. 18A-18F, in some examples, an implantable lead or medical device may have multiple tool-gripping portions (zones) and/or multiple non-gripping portions with at least some such non-gripping portions comprising an operative element 1464A (e.g. 64 in FIGS. 2A-2C). Such example arrangements may enable combining multiple sensors, allow for laparoscopic implantations, modification procedures, and/or support complex lead pathways in the body such as for deep brain stimulation.

As previously mentioned with regard to FIGS. 5A-5B, 6A-6C, 7-8, and 9A-9D, at least some aspects of example methods of the present disclosure may be performed via an implantable medical device (including but not limited to a lead in some examples) regardless of whether the operative element (e.g. 64 in FIGS. 2B-2C) of first portion (e.g. 60 in FIGS. 2B-2C or 360 in FIGS. 5A-5B) comprises a sensing element, stimulation element or other type of operative element. Moreover, via the principles of such example methods, other example implantations of a first portion (e.g. 60, 360) of an implantable medical device may be performed in other portions of the patient's body, different types of tissues, and/or for purposes other than obtaining respiratory information or treating respiratory issues.

With this in mind, FIG. 19A is diagram schematically representing an example method 1900 which comprises gripping, via a tool (e.g. 6A-9D, 14A-15C, other), a tool-gripping portion of an implantable medical device while positioning a first non-gripping portion of the implantable medical device into engagement relative to tissue of the patient's body. In some examples, the implantable medical device may comprise a stimulation lead and the first non-gripping portion may comprise a stimulation element, which may comprise at least one stimulation electrode. The stimulation element may comprise a paddle electrode, an array of ring electrodes, cuff electrode, etc. The method 1900 may comprise inserting the first non-gripping portion (e.g. distal portion including the stimulation element) within a subcutaneous region adjacent a target tissue, such as a nerve. By gripping the tool-gripping portion, one can better protect the first non-gripping portion during the insertion, handling, etc. In some examples, the nerve may be a hypoglossal nerve and the region in which the first non-gripping portion is inserted and implanted may be a head-and-neck region.

In some examples, the implantable medical device may take the form similar to that shown in FIG. 19B at 2000 in which the first non-gripping portion 2060 comprises a cuff electrode which is arranged distally of tool-gripping portion 2070 of lead 2052. As described in association with at least FIGS. 18A-18E, various combinations, spacing, etc. of non-gripping portions and tool-gripping portions may be implemented for device 2000 and/or the devices described below in association with FIGS. 19C-19E

As further shown in FIG. 19C, in some examples an implantable medical device 2100 which comprises at least some of substantially the same features and attributes as medical device 2000 (FIG. 19B), and which may comprise a cuff electrode 2160 including a non-gripping portion 2162, which comprises at least one electrode 2164 (e.g. three electrodes are shown) exposed on a body portion 2163. The cuff electrode 2610 comprises at least one flange, such as flanges 2168A, 2168B which are biased to be self-wrapping around a nerve to secure the electrodes 2164 relative to the nerve for stimulation. In some such examples, the tool-gripping portion 2070 is located adjacent and proximal to the cuff electrode 2160. In some such examples, the first and second flanges 2168A, 2168B also may be used in some instances as tool-gripping portions to help position the cuff electrode 2160 relative to tissues of interest, such as a nerve. The gripping of the flanges 2168A, 2168B may be used in a complementary manner with the gripping of tool-gripping portion 2070 (and/or other tool-gripping portions as described in the present disclosure) such that the tool may grip a flange (e.g. 2168A or 2168B), tool-gripping portion 2070, and/or simultaneously grip both a flange (e.g. 2168A or 2168B) and the tool-gripping portion 2070.

In some examples, the cuff electrode 2060 (FIG. 19B) or 2160 (FIG. 19C) may comprise at least some of substantially the same features and attributes as described in Bonde et al. U.S. Pat. No. 8,340,785, SELF EXPANDING ELECTRODE CUFF, issued on Dec. 25, 2102, Bonde et al. U.S. Pat. No. 9,227,053, SELF EXPANDING ELECTRODE CUFF, issued on Jan. 5, 2016, Johnson et al. U.S. Pat. No. 8,934,992, NERVE CUFF issued on Jan. 13, 2015, and/or Rondoni et al. CUFF ELECTRODE, WO 2019/032890 published on Feb. 14, 2019, each which are incorporated by reference herein in their entirety.

FIG. 19D is a diagram schematically representing an example device 2200. In some examples, device 2200 may comprise, and/or may be an example implementation of, at least some of substantially the same features and attributes as, the example methods and/or devices described in association with at least FIGS. 19A-19C. As shown in FIG. 19D, in some examples device 2200 may comprise a microstimulator 2260 including a tool-gripping portion 2270 (like 2070) formed on at least a portion of housing 2263. In some examples, device 2200 may comprise electrode(s) 2261 on a surface of housing 2263 and/or extending from housing 2263. The electrode(s) 2261 may comprise greater or fewer than the two shown in FIG. 19D, and the electrode(s) 2261 may comprise sensing electrodes and/or stimulation electrodes. As noted elsewhere, the tool-gripping portion 2270 may take one of many different forms, such as those described in association with FIGS. 18A-18F and/or in association with other examples of the present disclosure. In some examples, the microstimulator 2260 may comprise circuity, power (e.g. battery, rechargeable element, etc.), communication elements, etc. within housing 2263 for providing the stimulation and/or sensing of bodily tissues (e.g. nerve) and for communicating with elements external to the patient's body.

In general terms, the microstimulator 2260 is sized and/or shaped to be implanted subcutaneously, percutaneously, etc. via use of a tool (e.g. FIGS. 6A-9D, 14A-15B) without extensive tunneling within a body region and/or between multiple body regions. Accordingly, the microstimulator may be implanted with a neck region closely adjacent a target nerve (e.g. hypoglossal, phrenic, etc.) via use of the tool-gripping portion, which may facilitate initial positioning and/or re-positioning of the microstimulator 2260 relative to the target nerve while protecting the various components, functions of the microstimulator 2260.

In some examples, the microstimulator 2260 (and related elements) may comprise at least some of substantially the same features and attributes as described in association with at least MICROSTIMULATION SLEEP DISORDERED BREATHING (SDB) THERAPY DEVICE, published on May 26, 2017 as PCT Publication WO 2017/087681 from application PCT/US2016/062546 filed on Nov. 17, 2016, which is incorporated herein by reference.

FIG. 19E is a diagram of an example device 2300, which may comprise at least some of substantially the same features and attributes as example device 2200 (FIG. 19D), except comprising a lead portion 2352 extending from the microstimulator 2263, with the lead portion 2352 supporting cuff electrode 2060 (or cuff electrode 2160). It will be understood that electrode 2060 also may take other forms, such as a paddle electrode, ring electrode array, etc. As shown in FIG. 19E, the device 2300 comprises a tool-gripping portion 2370 on at least a portion of the lead portion 2352. However, at least a portion of the tool-gripping portion 2370 also may be present on the housing 2263 of the microstimulator 2260, present elsewhere, and/or otherwise arranged according to the general principles and specific examples previously described in association with FIGS. 18A-18E and/or other examples of the present disclosure.

Among other uses, the example implementations described in association with at least FIGS. 19A-19E may facilitate initial positioning and/or re-positioning an implantable medical device (including but not limited to a stimulation lead) during implantation relative to a nerve and/or other tissue.

Further examples are set forth below in the following numbered Feature Sets 1-92. It will be understood that any one or more of the example leads, devices, methods, etc. (and combinations thereof) recited in following numbered Feature Sets 1-92 may be implemented via at least some of substantially the same features and attributes of the examples previously described in association with at least FIGS. 1-19E and generally within the present disclosure.

Feature Set 1: An implantable lead comprises a first non-gripping portion and a first tool-gripping portion.

Feature Set 2: The implantable lead of Feature Set 1, wherein the first tool-gripping portion is proximally adjacent the first non-gripping portion.

Feature Set 3: The implantable lead of Feature Set 1, wherein the first non-gripping portion comprises a distal portion of the lead.

Feature Set 4: The implantable lead of Feature Set 1, wherein the first tool-gripping portion is spaced apart from the first non-gripping portion.

Feature Set 5: The implantable lead of Feature Set 1, wherein the first tool-gripping portion is immediately adjacent the first non-gripping portion.

Feature Set 6: The implantable lead of Feature Set 1, further comprising a plurality of tool-gripping portions, including the first tool-gripping portion.

Feature Set 7: The implantable lead of Feature Set 6, further comprising the first non-gripping portion being interposed between two tool-gripping portions of the plurality of tool-gripping portions.

Feature Set 8: The implantable lead of Feature Set 6, further comprising a plurality of non-gripping portions, including the first non-gripping portion.

Feature Set 9: The implantable lead of Feature Set 1, comprising a plurality of non-gripping portions, including the first non-gripping portion.

Feature Set 10: The implantable lead of Feature Set 1, wherein the first tool-gripping portion comprises a first visual designator.

Feature Set 11: The implantable lead of Feature Set 10, wherein the first visual designator comprises at least one of a color, texture, surface pattern, absorbance, reflectance, relative opaqueness, relative radiopaqueness, topographic feature, and profile.

Feature Set 12: The implantable lead of Feature Set 10, wherein the first non-gripping portion comprises a second visual designator visually perceptibly different from the first visual designator.

Feature Set 13: The implantable lead of Feature Set 12, wherein the second visual designator comprises at least one of a color, texture, surface pattern, absorbance, reflectance, relative opaqueness, relative radiopaqueness, topographic feature, and profile.

Feature Set 14: The implantable lead of Feature Set 11, wherein the first non-gripping portion comprises an operative element.

Feature Set 15: The implantable lead of Feature Set 1, wherein the operative element comprises at least one of a mechanical function element, an electrical function element, and a chemical function element.

Feature Set 16: The implantable lead of Feature Set 1, wherein the operative element comprises at least one of a sensing element and a stimulation element.

Feature Set 17: The implantable lead of Feature Set 16, wherein the sensing element comprises a pressure-indicative sensor.

Feature Set 18: The implantable lead of Feature Set 16, wherein the stimulation element comprises at least one surface electrode.

Feature Set 19: The implantable lead of Feature Set 1, comprising: a first anchor portion proximal to the first tool-gripping portion, wherein the first tool-gripping portion is interposed between the first non-gripping portion and the anchor portion.

Feature Set 20: The implantable lead of Feature Set 19, wherein the first anchor portion comprises a separate element coupled relative to a body of the lead.

Feature Set 21: The implantable lead of Feature Set 19, wherein the first anchor portion is integrated into a body of the lead.

Feature Set 22: The implantable lead of Feature Set 21, wherein the first anchor portion comprises an external surface defining a profile which substantially matches a profile of an external surface of adjacent portions of the lead body.

Feature Set 23: The implantable lead of Feature Set 21, wherein the first anchor portion comprises suture holes located at least partially interior to the outer profile of the first anchor portion.

Feature Set 24: The implantable lead of Feature Set 1, wherein the first tool-gripping portion comprises a first visual designator, and further comprising: an anchor portion coupled to the lead and comprising a third visual designator, wherein the first color of the first visual designator of the first tool-gripping portion is a different color than the third visual designator of the anchor portion.

Feature Set 25: The implantable lead of Feature Set 1, wherein the first non-gripping portion comprises an unexposed proximal segment, and wherein the first tool-gripping portion comprises a distal portion coextensive with, and overlapping, at least the unexposed proximal segment of the first non-gripping portion.

Feature Set 26: The implantable device of Feature Set 25, wherein the distal portion of the tool-gripping portion comprises a first electrically conductive inner portion and a first electrically non-conductive outer portion covering the first electrically conductive inner portion, wherein the first electrically non-conductive outer portion comprises a first thickness.

Feature Set 27: The implantable lead of Feature Set 26, wherein the unexposed proximal segment of the first non-gripping portion at least partially defines the first electrically conductive inner portion of the distal portion of the tool-gripping portion.

Feature Set 28: The implantable lead of Feature Set 25, wherein the tool-gripping portion comprises a proximal portion adjacent the anchor portion.

Feature Set 29: The implantable lead of Feature Set 28, wherein the proximal portion of the tool-gripping portion comprises a second electrically conductive inner portion and a second electrically non-conductive outer portion covering the second electrically conductive inner portion, wherein the second electrically non-conductive outer portion comprises a third thickness which is substantially greater than a diameter of the second non-conductive inner portion of the proximal portion of the visually designated tool-gripping portion.

Feature Set 30: The implantable lead of Feature Set 26, wherein the first electrically conductive inner portion comprises the unexposed proximal segment of the first non-gripping portion, which comprises an elongate tube including an outer surface having a first recess sized and shaped to receive a complementary first protrusion on a distal inner surface of the first electrically non-conductive outer portion.

Feature Set 31: The implantable lead of Feature Set 1, wherein the tool-gripping portion comprises an anchor.

Feature Set 32: The implantable lead of Feature Set 11, further comprising a first anchor portion proximal to the tool-gripping portion.

Feature Set 33: The implantable lead of Feature Set 32, wherein a body of the lead comprises a serpentine-shaped portion proximal to at least the first anchor portion.

Feature Set 34: The implantable lead of Feature Set 33, further comprising a second anchor portion coupled to the lead body proximal to, and spaced apart from, the first anchor portion, and wherein the serpentine-shaped portion of the lead body is proximal to the second anchor portion.

Feature Set 35: The implantable lead of Feature Set 32, wherein the first anchor portion comprises an array of topographic features on a second face opposite a first face of the distal operative segment from which a sensor is oriented to face tissue.

Feature Set 36: The implantable device of Feature Set 35, wherein the first anchor portion comprises a body and a pair of arms extending outward from opposite sides of the body of the first anchor portion, wherein the array of topographic features are located at least partially on the body and at least partially on the respective arms.

Feature Set 37: An internally deliverable medical device comprising an elongate, flexible resilient body including a non-gripping portion and a tool-gripping portion.

Feature Set 38: The internally deliverable medical device of Feature Set 37, wherein the non-gripping portion comprises a distal portion of the medical device and the tool-gripping portion extends proximally adjacent the non-gripping portion.

Feature Set 39: The internally deliverable medical device of Feature Set 37, further comprising a catheter including the elongate flexible resilient body.

Feature Set 40: The internally deliverable medical device of Feature Set 39, wherein the non-gripping portion comprises a drug delivery portion.

Feature Set 41: The internally deliverable medical device of Feature Set 38, further comprising an implantable lead including the elongate flexible resilient body and wherein the non-gripping portion comprises a sensing element.

Feature Set 42: The internally deliverable medical device of Feature Set 37, further comprising an implantable lead including the elongate flexible resilient body and wherein the non-gripping portion comprises a stimulation element.

Feature Set 43: The internally deliverable medical device of Feature Set 37, wherein the tool-gripping portion comprises a first visual designator.

Feature Set 44: The internally deliverable medical device of Feature Set 43, wherein the non-gripping portion comprises a second visual designator different from the first visual designator.

Feature Set 45: The internally deliverable medical device of Feature Set 44, further comprising a first anchor proximally adjacent the tool-gripping portion.

Feature Set 46: The internally deliverable medical device of Feature Set 45, wherein the first anchor comprises a third visual designator different from at least the first visual designator.

Feature Set 47. A method comprising arranging an internally deliverable medical device to include a first non-gripping portion and a first tool-gripping portion which includes a first visual designator.

Feature Set 48: The method of Feature Set 47, further comprising arranging the first non-gripping portion to be distal to the first tool-gripping portion, which extends proximally from the first non-gripping portion.

Feature Set 49: The method of Feature Set 47, further comprising arranging the internally deliverable medical device to comprise an implantable lead including, at the non-gripping portion, at least one of a sensing element and a stimulation element.

Feature Set 50: The method of Feature Set 47, further comprising arranging the internally deliverable medical device to comprise a catheter including a drug delivery component within the non-gripping portion.

Feature Set 51: The method of Feature Set 47, further comprising arranging the internally deliverable medical device to include an elongate, flexible resilient body, which includes at least a portion of the first tool-gripping portion.

Feature Set 52: The method of Feature Set 51, further comprising arranging the first tool-gripping portion to include a proximal electrically non-conductive outer portion and a distal electrically non-conductive outer portion, wherein the proximal electrically non-conductive outer portion has a second thickness substantially greater than a first thickness of the distal electrically non-conductive outer portion.

Feature Set 53: The method of Feature Set 47, further comprising arranging the first non-gripping portion to include a second visual designator perceptibly different from the first visual designator of the first tool-gripping portion.

Feature Set 54: The method of Feature Set 53, wherein each of the respective first and second visual designators comprise at least one of a color, texture, reflectance, absorbance, surface pattern, relative opacity, relative radiopaqueness, topographic feature, and profile.

Feature Set 55: The method of Feature Set 47, further comprising arranging a first anchor portion proximally adjacent the first tool-gripping portion.

Feature Set 56: The method of Feature Set 55, further comprising arranging the first anchor portion to include a third visual designator different from at least the first visual designator.

Feature Set 57: The method of Feature Set 56, further comprising arranging the first anchor portion as a tool-grippable element.

Feature Set 58: The method of Feature Set 47, further comprising: arranging the first non-gripping portion to be distally adjacent the first tool-gripping portion; arranging the first non-gripping portion to include an unexposed proximal segment; and arranging the distal electrically non-conductive outer portion of the first tool-gripping portion to overlap with the unexposed proximal segment of the first non-gripping portion.

Feature Set 59: The method of Feature Set 47, further comprising arranging the first thickness and the second thickness to be substantially greater than a third thickness of an electrically non-conductive outer portion of the first non-gripping portion.

Feature Set 60: The method of Feature Set 47, comprising gripping, via a tool, the tool-gripping portion of the implantable lead and maintaining the grip while inserting and advancing the first non-gripping portion of the lead into and through an incision and subcutaneously within tissue.

Feature Set 61: The method of Feature Set 60, further comprising performing the gripping while not visualizing at least the first non-gripping portion during subcutaneous advancement of the medical device.

Feature Set 62: The method of Feature Set 60, further comprising evaluating subcutaneous advancement of the first non-gripping portion, at least partially based on, a position of a proximal portion of the first tool-gripping portion relative to at least the incision.

Feature Set 63: The method of Feature Set 62, further comprising: upon a determination to further distally, subcutaneously advance the first non-gripping portion, releasing the tool from the first tool-gripping portion and re-gripping the tool on an exposed, more proximal portion of the first tool-gripping portion; and while maintaining the re-gripped position, further subcutaneously advancing the first non-gripping portion without visualizing the first non-gripping portion.

Feature Set 64: The method of Feature Set 60, comprising arranging the tool to include a pair of opposed gripping portions.

Feature Set 65: The method of Feature Set 64, comprising arranging the tool to include a pivot mechanism to permit selective movement of the opposed gripping portions toward each other for gripping the tool-gripping portion of the medical device.

Feature Set 66: The method of Feature Set 60, further comprising arranging the opposed gripping portions of the tool to include a second arcuate cross-Feature Setal shape generally corresponding to a first arcuate cross-Feature Setal shape of an outer surface of the first tool-gripping portion of the medical device.

Feature Set 67: The method of Feature Set 66, wherein the opposed gripping portions of the tool each have a length at least one-half a length of the tool-gripping portion. Feature Set 68: The method of Feature Set 60, further comprising arranging the opposed gripping portions of the tool as elongate elements which are spaced apart from each other in a generally parallel relationship and by a first distance approximately the same as an outer cross-Feature Setal dimension of the tool-gripping portion.

Feature Set 69: The method of Feature Set 68, wherein performing the gripping of the first tool-gripping portion of the medical device comprises positioning the elongate elements to be transverse to a longitudinal axis of the tool-gripping portion, and maneuvering the elongate elements to induce a friction fit engagement against an outer surface of the first tool-gripping portion of the medical device.

Feature Set 70: The method of Feature Set 60, further comprising: evaluating the distal subcutaneous advancement of the first non-gripping portion, at least partially based on, a position of the more proximal portion of the first tool-gripping portion relative to the incision.

Feature Set 71: The method of Feature Set 60, further comprising: arranging an anchor proximal to the first tool-gripping portion of the medical device and arranging an array of topographic features on a first surface of at least a body of the anchor; during subcutaneous advancement of the first non-gripping portion, orienting an operative component of the first non-gripping portion relative to a target tissue while maintaining the topographic features to face outwardly away from body; and placing a finger on at least some of the topographic features while advancing the first non-gripping portion toward a target tissue portion.

Feature Set 72: A method of manufacturing comprising: arranging an internally deliverable medical device to include a first non-gripping portion; and arranging a body of the medical device to extend proximally from the first portion, and including a first tool-gripping portion proximally adjacent the non-gripping portion.

Feature Set 73: The method of Feature Set 72, further comprising arranging the first tool-gripping portion to include a first visual designator.

Feature Set 74: The method of Feature Set 73, further comprising arranging the first non-gripping portion to include a second visual designator visually perceptibly different from the first visual designator.

Feature Set 75: The method of Feature Set 73, further comprising arranging the first visual designator to include at least one of a color, a texture, a surface pattern, absorbance, reflectance, a relative opaqueness, a relative radiopaqueness, a topographical feature, and a profile.

Feature Set 76: The method of Feature Set 72, comprising arranging the first non-gripping portion to include at least one of a mechanical function element, an electrical function element, and a chemical function element.

Feature Set 77: The method of Feature Set 72, further comprising arranging the operative element to include at least one of a sensing element and a stimulation element.

Feature Set 78: The method of Feature Set 77, further comprising arranging the sensing element to include a pressure-indicative sensor.

Feature Set 79: The method of Feature Set 77, wherein the stimulation element comprises at least one surface electrode.

Feature Set 80: The method of Feature Set 72, comprising: fixing a first anchor portion relative to the body at a location proximal to the first tool-gripping portion; and arranging the first tool-gripping portion to be interposed between the first non-gripping portion and the first anchor portion.

Feature Set 81: The method of Feature Set 72, further comprising: arranging the first tool-gripping portion to include a first visual designator; fixing a first anchor portion relative to the body proximal to first tool-gripping portion; and arranging the first anchor portion to include a third visual designator, wherein the first color of the first visual designator of the first tool-gripping portion is a different color from the third visual designator of the first anchor portion.

Feature Set 82: The method of Feature Set 72, comprising: arranging the first non-gripping portion to include an unexposed proximal segment; and arranging the first tool-gripping portion to include a distal portion coextensive with, and overlapping, at least the unexposed proximal segment.

Feature Set 83: The method of Feature Set 82, further comprising arranging the distal portion of the first tool-gripping portion to include a first electrically conductive inner portion and a first electrically non-conductive outer portion covering the first electrically conductive inner portion, wherein the first electrically non-conductive outer portion comprises a first thickness.

Feature Set 84: The method of Feature Set 82, further comprising arranging the first tool-gripping portion to include a proximal portion adjacent the first anchor portion.

Feature Set 85: The method of Feature Set 84, further comprising arranging the proximal portion of the first tool-gripping portion to include a second electrically conductive inner portion and a second electrically non-conductive outer portion covering the second electrically conductive inner portion.

Feature Set 86: The method of Feature Set 85, wherein the second electrically non-conductive outer portion comprises a third thickness which is substantially greater than a diameter of the second non-conductive inner portion of the proximal portion of the tool-gripping portion.

Feature Set 87: The method of Feature Set 82, further comprising: arranging the first electrically conductive inner portion as the unexposed proximal segment of the first non-gripping portion; and arranging the unexposed proximal segment of the first non-gripping portion to include an elongate tube including an outer surface having a first recess sized and shaped to receive a complementary first protrusion on a distal inner surface of the first electrically non-conductive outer portion.

Feature Set 87: The method of Feature Set 82, further comprising: arranging the first electrically conductive inner portion as the unexposed proximal segment of the first non-gripping portion; and arranging the unexposed proximal segment of the first non-gripping portion to include an elongate tube including an outer surface having a first recess sized and shaped to receive a complementary first protrusion on a distal inner surface of the first electrically non-conductive outer portion.

Feature Set 87: The method of Feature Set 82, further comprising: arranging the first electrically conductive inner portion as the unexposed proximal segment of the first non-gripping portion; and arranging the unexposed proximal segment of the first non-gripping portion to include an elongate tube including an outer surface having a first recess sized and shaped to receive a complementary first protrusion on a distal inner surface of the first electrically non-conductive outer portion.

Feature Set 90: The method of Feature Set 89, further comprising: fixing a second anchor portion relative to the body proximal to, and spaced apart from, the first anchor portion, and arranging the serpentine-shaped portion of the body to be located proximal to the second anchor portion.

Feature Set 91: The method of Feature Set 88, further comprising arranging the first anchor portion to include an array of topographic features on a second face of the lead body which is opposite a first face of the body from which a sensor is oriented to face tissue.

Feature Set 92: The method of Feature Set 91, further comprising arranging the first anchor portion to include a body and a pair of arms extending outward from opposite sides of the body of the first anchor portion, wherein the array of topographic features are located at least partially on the body and at least partially on the respective arms.

Although specific examples have been illustrated and described herein, a variety of alternate and/or equivalent implementations may be substituted for the specific examples shown and described without departing from the scope of the present disclosure. This application is intended to cover any adaptations or variations of the specific examples discussed herein.

1. An implantable medical device comprising:
an elongate, flexible resilient body including a non-gripping portion and a tool-gripping portion.

2. The implantable medical device of claim 1, comprising:
an implantable lead including the elongate flexible resilient body.

3. The implantable medical device of claim 2, wherein the elongate, flexible resilient body includes at least a portion of the first tool-gripping portion.

4. The implantable medical device of claim 2, wherein the non-gripping portion comprises a sensing element.

5. The implantable medical device of claim 2, wherein the non-gripping portion comprises a stimulation element.

6. The implantable medical device of claim 4, wherein the stimulation element comprises a cuff electrode.

7. The implantable medical device of claim 1, wherein the tool-gripping portion comprises a first visual designator.

8. The implantable medical device of claim 7, wherein the first visual designator comprises at least one of a color, texture, surface pattern, absorbance, reflectance, relative opaqueness, relative radiopaqueness, topographic feature, and profile.

9. The implantable medical device of claim 7, wherein the first non-gripping portion comprises a second visual designator visually perceptibly different from the first visual designator, wherein the second visual designator optionally comprises at least one of a color, texture, surface pattern, absorbance, reflectance, relative opaqueness, relative radiopaqueness, topographic feature, and profile.

10. The implantable medical device of claim 1, wherein the first non-gripping portion comprises an operative element.

11. The implantable medical device of claim 1, wherein the operative element comprises at least one of a sensing element and a stimulation element, and optionally comprising the sensing element comprising a pressure-indicative sensor.

12. The implantable medical device of claim 10, wherein the operative element comprises at least one of a mechanical function element, an electrical function element, and a chemical function element.

13. The implantable medical device of claim 1, wherein the tool-gripping portion comprises a plurality of spaced apart tool-gripping portions.

14. A method comprising:
arranging an internally deliverable medical device to include a first non-gripping portion and a first tool-gripping portion which includes a first visual designator.

15. The method of claim 14, comprising:
arranging the internally deliverable medical device to include an elongate, flexible resilient body, which includes at least a portion of the first tool-gripping portion.

16. The method of claim 15, comprising:
arranging the first tool-gripping portion to include a proximal electrically non-conductive outer portion and a distal electrically non-conductive outer portion, wherein the proximal electrically non-conductive outer portion has a second thickness substantially greater than a first thickness of the distal electrically non-conductive outer portion.

17. The method of claim 14, comprising:
gripping, via a tool, the tool-gripping portion of the implantable lead and maintaining the grip while inserting and advancing the first non-gripping portion of the lead into and through an incision and subcutaneously within tissue.

18. The method of claim 14, comprising
gripping, via a tool, the tool-gripping portion of an implantable medical device while inserting and positioning a first non-gripping portion of the device into engagement relative to tissue within the patient's body.

19. The method of claim 18, comprising:
arranging the first non-gripping portion as a cuff electrode including at least one stimulation electrode.

20. The method of claim 18, comprising:
arranging the first non-gripping portion as a microstimulator.
